SEQUENCE LISTING (60 sequences)

SEQ ID NO: 1 - DNA, 7179 bp, Artificial
  pIM-Universal-TK-Neo
gctagggataacagggtaatatcgcgccagatctgtacattcgaagatatcttaattaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaggccggcctgcaggttcgaaaagggcgaattcgcgtttaaagcttgtacatcgatgcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgtaagtgatatctactagatttatcaaaaagagtgttgacttgtgagcgctcacaattgatacttagattcatcgagagggacacgtcgactactaaccttcttctctttcctacagctgagatcaccggcgaaggagggccaccatggcttcttaccctggacaccagcatgcttctgcctttgaccaggctgccagatccaggggccactccaacaggagaactgccctaagacccagaagacagcaggaagccactgaggtgaggcctgagcagaagatgccaaccctgctgagggtgtacattgatggacctcatggcatgggcaagaccaccaccactcaactgctggtggcactgggctccagggatgacattgtgtatgtgcctgagccaatgacctactggagagtgctaggagcctctgagaccattgccaacatctacaccacccagcacaggctggaccagggagaaatctctgctggagatgctgctgtggtgatgacctctgcccagatcacaatgggaatgccctatgctgtgactgatgctgttctggctcctcacattggaggagaggctggctcttctcatgcccctccacctgccctgaccctgatctttgacagacaccccattgcagccctgctgtgctacccagcagcaaggtacctcatgggctccatgaccccacaggctgtgctggcttttgtggccctgatccctccaaccctccctggcaccaacattgttctgggagcactgcctgaagacagacacattgacaggctggcaaagaggcagagacctggagagagactggacctggccatgctggctgcaatcagaagggtgtatggactgctggcaaacactgtgagatacctccagtgtggaggctcttggagagaggactggggacagctctctggaacagcagtgccccctcaaggagctgagccccagtccaatgctggtccaagaccccacattggggacaccctgttcaccctgttcagagcccctgagctgctggctcccaatggagacctgtacaatgtgtttgcctgggctctggatgttctagccaagaggctgaggtccatgcatgtgttcatcctggactatgaccagtcccctgctggatgcagagatgctctgctgcaactaacctctggcatggtgcagacccatgtgaccacccctggcagcatccccaccatctgtgacctagccagaacctttgccagggagatgggagaggccaactaaacctgagctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatcatttaaatgttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggatctcgagcggccgcggcgcgccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgattaattaacaggactgaccgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgacgcgaattcgcccttgcgcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagccttaggcgcg

SEQ ID NO: 2 - DNA, 7415 bp, Artificial
  pIM-Universal-CDUPRT-Hygro
gctagggataacagggtaatatcgcgccagatctgtacattcgaagatatcttaattaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaggccggcctgcaggttcgaaaagggcgaattcgcgtttaaagcttgtacatcgatgcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctggtgggtagggatgagggagggaggggcattgtgatgtacagggctgctctgtgagatcaagggtctcttaagggtgggagctggggcagggactacgagagcagccagatgggctgaaagtggaactcaaggggtttctggcacctacctacctgcttcccgctggggggtggggagttggcccagagtcttaagattggggcagggtggagaggtgggctcttcctgcttcccactcatcttatagctttctttccccagatccgaattcgagatccaaaccaaggaggaaaggatatcacagaggagaccatggtcacaggaggcatggcttcaaagtgggaccagaagggcatggacattgcctatgaggaggctgctctgggctacaaggagggaggggtcccaattggtggctgcctcatcaacaacaaggatggcagtgtcctgggcaggggccacaacatgaggttccagaagggcagtgccaccctgcatggggagatcagcaccctggagaactgtggcaggctggagggcaaggtctacaaggacaccactctgtacaccaccctcagcccttgtgacatgtgcacaggggccatcatcatgtatggcattcccaggtgtgtggtgggagagaatgtcaacttcaagtcaaaaggagagaagtacctccagaccaggggccatgaggtggttgtggtggatgatgagaggtgcaagaagattatgaagcagttcattgatgagagaccccaggactggtttgaggacattggggaggcctctgagcccttcaagaatgtgtacctcctcccccagaccaaccaactcctgggactctacaccatcatcaggaacaagaacaccaccaggccagacttcatcttctacagtgacaggatcatcaggctcctggtggaggagggcctcaaccacctccctgtgcagaagcagattgtggagactgacaccaatgagaactttgagggagtgtctttcatgggcaagatttgtggggtgtccattgtgagggctggggagagcatggagcagggcctgagggactgttgcaggagtgtgaggattggcaagatcctgatccagagggatgaggagactgccctgcccaagctgttctatgagaagctccctgaagacatctctgagaggtatgtcttcctcctggaccccatgctggcaactggaggctctgcaatcatggccactgaggtgctcatcaagaggggagtcaagcctgagaggatctacttcctcaacctcatctgctcaaaggagggcattgagaagtaccatgctgccttccctgaagtgaggattgtcactggggctctggacaggggcctggatgagaacaagtacctggtccctggcctgggagactttggggacagatactactgtgtctaaacctgagctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatccatttaaatgttaattaaaaacccgcttcggcgggtttttttatgcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggatctcgataacaaaaaaccccgccccggcggggttttttgttagcggccgcggcgcgccgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggagaagggcgaattctgcagatgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctacgcgaattcgcccttgcgcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagccttaggcgcg

SEQ ID NO: 3 - DNA, 795 bp, Artificial
  nptl gene
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 4 - DNA, 1038 bp, Artificial
  hph gene
atgaagaagcccgaactcaccgctaccagcgttgaaaaatttctcatcgagaagttcgacagtgtgagcgacctgatgcagttgtcggagggcgaagagagccgagccttcagcttcgatgtcggcggacgcggctatgtactgcgggtgaatagctgcgctgatggcttctacaaagaccgctacgtgtaccgccacttcgccagcgctgcactacccatccccgaagtgttggacatcggcgagttcagcgagagcctgacatactgcatcagtagacgcgcccaaggcgttactctccaagacctccccgaaacagagctgcctgctgtgttacagcctgtcgccgaagctatggatgctattgccgccgccgacctcagtcaaaccagcggcttcggcccattcgggccccaaggcatcggccagtacacaacctggcgggatttcatttgcgccattgctgatccccatgtctaccactggcagaccgtgatggacgacaccgtgtccgccagcgtagctcaagccctggacgaactgatgctgtgggccgaagactgtcccgaggtgcgccacctcgtccatgccgacttcggcagcaacaacgtcctgaccgacaacggccgcatcaccgccgtaatcgactggtccgaagctatgttcggggacagtcagtacgaggtggccaacatcttcttctggcggccctggctggcttgcatggagcagcagactcgctacttcgagcgccggcatcccgagctggccggcagccctcgtctgcgagcctacatgctgcgcatcggcctggatcagctctaccagagcctcgtggacggcaacttcgacgatgctgcctgggctcaaggccgctgcgatgccatcgtccgcagcggggccggcaccgtcggtcgcacacaaatcgctcgccggagcgcagccgtatggaccgacggctgcgtcgaggtgctggccgacagcggcaaccgccggcccagtacacgaccgcgcgctaaggaggtaggtcgagtttaa

SEQ ID NO: 5 - DNA, 600 bp, Artificial
  pac
atgaccgagtacaagcctaccgtgcgcctggccactcgcgatgatgtgccccgcgccgtccgcactctggccgccgctttcgccgactaccccgctacccggcacaccgtggaccccgaccggcacatcgagcgtgtgacagagttgcaggagctgttcctgacccgcgtcgggctggacatcggcaaggtgtgggtagccgacgacggcgcggccgtggccgtgtggactacccccgagagcgttgaggccggcgccgtgttcgccgagatcggcccccgaatggccgagctgagcggcagccgcctggccgcccagcagcaaatggagggcctgcttgccccccatcgtcccaaggagcctgcctggtttctggccactgtaggagtgagccccgaccaccagggcaagggcttgggcagcgccgtcgtgttgcccggcgtagaggccgccgaacgcgccggtgtgcccgcctttctcgaaacaagcgcaccaagaaaccttccattctacgagcgcctgggcttcaccgtgaccgccgatgtcgaggtgcccgagggacctaggacctggtgtatgacacgaaaacctggcgcctaa

SEQ ID NO: 6 - DNA, 399 bp, Artificial
  bsr gene
atggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaa

SEQ ID NO: 7 - DNA, 375 bp, Artificial
  sh ble gene
atggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactga

SEQ ID NO: 8 - DNA, 655 bp, Artificial
  HSV TK DelCpG gene
cgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatt

SEQ ID NO: 9 - DNA, 344 bp, Artificial
  CDUPRT DelCpG
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagct

SEQ ID NO: 10 - DNA, 1131 bp, Artificial
  cytomegalovirus immediate-early promoter
atggcttcttaccctggacaccagcatgcttctgcctttgaccaggctgccagatccaggggccactccaacaggagaactgccctaagacccagaagacagcaggaagccactgaggtgaggcctgagcagaagatgccaaccctgctgagggtgtacattgatggacctcatggcatgggcaagaccaccaccactcaactgctggtggcactgggctccagggatgacattgtgtatgtgcctgagccaatgacctactggagagtgctaggagcctctgagaccattgccaacatctacaccacccagcacaggctggaccagggagaaatctctgctggagatgctgctgtggtgatgacctctgcccagatcacaatgggaatgccctatgctgtgactgatgctgttctggctcctcacattggaggagaggctggctcttctcatgcccctccacctgccctgaccctgatctttgacagacaccccattgcagccctgctgtgctacccagcagcaaggtacctcatgggctccatgaccccacaggctgtgctggcttttgtggccctgatccctccaaccctccctggcaccaacattgttctgggagcactgcctgaagacagacacattgacaggctggcaaagaggcagagacctggagagagactggacctggccatgctggctgcaatcagaagggtgtatggactgctggcaaacactgtgagatacctccagtgtggaggctcttggagagaggactggggacagctctctggaacagcagtgccccctcaaggagctgagccccagtccaatgctggtccaagaccccacattggggacaccctgttcaccctgttcagagcccctgagctgctggctcccaatggagacctgtacaatgtgtttgcctgggctctggatgttctagccaagaggctgaggtccatgcatgtgttcatcctggactatgaccagtcccctgctggatgcagagatgctctgctgcaactaacctctggcatggtgcagacccatgtgaccacccctggcagcatccccaccatctgtgacctagccagaacctttgccagggagatgggagaggccaactaa

SEQ ID NO: 11 - DNA, 1122 bp, Artificial
  simian virus 40 promoter
atggtcacaggaggcatggcttcaaagtgggaccagaagggcatggacattgcctatgaggaggctgctctgggctacaaggagggaggggtcccaattggtggctgcctcatcaacaacaaggatggcagtgtcctgggcaggggccacaacatgaggttccagaagggcagtgccaccctgcatggggagatcagcaccctggagaactgtggcaggctggagggcaaggtctacaaggacaccactctgtacaccaccctcagcccttgtgacatgtgcacaggggccatcatcatgtatggcattcccaggtgtgtggtgggagagaatgtcaacttcaagtcaaaaggagagaagtacctccagaccaggggccatgaggtggttgtggtggatgatgagaggtgcaagaagattatgaagcagttcattgatgagagaccccaggactggtttgaggacattggggaggcctctgagcccttcaagaatgtgtacctcctcccccagaccaaccaactcctgggactctacaccatcatcaggaacaagaacaccaccaggccagacttcatcttctacagtgacaggatcatcaggctcctggtggaggagggcctcaaccacctccctgtgcagaagcagattgtggagactgacaccaatgagaactttgagggagtgtctttcatgggcaagatttgtggggtgtccattgtgagggctggggagagcatggagcagggcctgagggactgttgcaggagtgtgaggattggcaagatcctgatccagagggatgaggagactgccctgcccaagctgttctatgagaagctccctgaagacatctctgagaggtatgtcttcctcctggaccccatgctggcaactggaggctctgcaatcatggccactgaggtgctcatcaagaggggagtcaagcctgagaggatctacttcctcaacctcatctgctcaaaggagggcattgagaagtaccatgctgccttccctgaagtgaggattgtcactggggctctggacaggggcctggatgagaacaagtacctggtccctggcctgggagactttggggacagatactactgtgtctaa

SEQ ID NO: 12 - DNA, 883 bp, Artificial
  human elongation factor 1  promoter
gatctaaagctaactgtaggactgagtctattctaaactgaaagcctggacatctggagtaccagggggagatgacgtgttacgggcttccataaaagcagctggctttgaatggaaggagccaagaggccagcacaggagcggattcgtcgctttcacggccatcgagccgaacctctcgcaagtccgtgagccgttaaggaggcccccagtcccgacccttcgccccaagcccctcggggtccccgggcctggtactccttgccacacgggaggggcgcggaagccggggcggaggaggagccaaccccgggctgggctgagacccgcagaggaagacgctctagggatttgtcccggactagcgagatggcaaggctgaggacgggaggctgattgagaggcgaaggtacaccctaatctcaatacaacctttggagctaagccagcaatggtagagggaagattctgcacgtcccttccaggcggcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgtt

SEQ ID NO: 13 - DNA, 301 bp, Artificial
  human phosphoglycerate kinase promoter
aattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaa

SEQ ID NO: 14 - DNA, 507 bp, Artificial
  murine phosphoglycerate kinase promoter
ctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgac

SEQ ID NO: 15 - DNA, 1212 bp, Artificial
  human polyubiquitin promoter
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagac

SEQ ID NO: 16 - DNA, 753 bp, Artificial
  thymidine kinase promoter from human herpes simplex virus
aaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaa

SEQ ID NO: 17 - DNA, 746 bp, Artificial
  human growth arrest specific 5 promoter
ggcaagggagttgcgggcgcacaggtaaggcgcgggaccgggaagggggcggcgccccgggatcggtctagggcggcggaggctgccggggcgggggtgtgtctgggtgtggccccggcgcgcgcggggtcctgagggaggggctcgccaagggggcgggcccgcgagctcgacagccccgagccaaaggggcggaaggtcgccgagtgctgggaggggaggtggggcagcgactgagcgccgcaggagctggctgcaacccagacgcggcccgggagcgtcgggtatgagctaacggggcgggggtgcggggaacgagcccagtcaaggagcagcacctacctccgggggtggaaatgggccggaatgggccggaacaccgtcccggaagtgaaaccccccgccccctcctccgcagcgagcgacgtgccggaaggaaatcagtcaccctccccccgcccctcccccagcactttccttgcaggagccccctcccctcagcctgtactcctcagggaggcggagggcgggcctatcgtgcccgcggcaactccgccctccgggctctcgggggcgtggccagagggaaagttttgtgggcctgaagaaggtggggcttgaggaggagtctgagggcgtgtgaggggcggggtttaagtggacgcagcaagcagctttgcgtcttgacgtcacgaagaccttatatactcgactcagccgccatctcagcctttcggagctgtgcg

SEQ ID NO: 18 - DNA, 316 bp, Artificial
  tetracycline-responsive element
cgagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgcc

SEQ ID NO: 19 - DNA, 596 bp, Artificial
  internal ribosomal entry site (IRES) sequence from encephalopathy myocarditis virus
aattccgcccctctcccccccccccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataa

SEQ ID NO: 20 - DNA, 469 bp, Artificial
  IRES sequence from foot and mouth disease virus
agcaggtttccccaatgacacaaaacgtgcaacttgaaactccgcctggtctttccaggtctagaggggtaacactttgtactgcgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttgcaattactgaccctatgaataca

SEQ ID NO: 21 - DNA, 131 bp, Artificial
  SV40 polyadenylation signal
aacttgtttattgcagcttataatggttacaaataaagcaatagcatcacaaatttcacaaataaagcatttttttcactgcattctagttgtggtttgtccaaactcatcaatgtatcttatcatgtctg

SEQ ID NO: 22 - DNA, 215 bp, Artificial
  bovine growth hormone polyadenylation signal
gcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 23 - protein, 8 aa, Artificial
  FLAG tag
DYKDDDDK

SEQ ID NO: 24 - protein, 6 aa, Artificial
  FLASH/REASH tag
CCPGCC

SEQ ID NO: 25 - protein, 7 aa, Artificial
  IQ tag
IQSPHFF

SEQ ID NO: 26 - protein, 6 aa, Artificial
  histidine tag
HHHHHH

SEQ ID NO: 27 - protein, 8 aa, Artificial
  STREP tag
WSHPEFEK

SEQ ID NO: 28 - protein, 45 aa, Artificial
  streptavidin binding protein
MDEKTTGWRGGHVVEGLAGELEQLRARLEHHPQGQREPSGGCKLG

SEQ ID NO: 29 - protein, 26 aa, Artificial
  calmodulin binding protein
KRRWKKNFIAVSAANRFKKISSSGAL

SEQ ID NO: 30 - protein, 9 aa, Artificial
  haemagglutinin tag
YPYDVPDYA

SEQ ID NO: 31 - protein, 10 aa, Artificial
  c-myc tag
EQKLISEEDL

SEQ ID NO: 32 - protein, 14 aa, Artificial
  V5 tag
GKPIPNPLLGLDST

SEQ ID NO: 33 - protein, 7 aa, Artificial
  nuclear localization signal from nucleoplasmin
PKKKRKV

SEQ ID NO: 34 - protein, 16 aa, Artificial
  NLS from SV40
KRPAATKKAGQAKKKK

SEQ ID NO: 35 - protein, 6 aa, Artificial
  NLS consensus
KKRXKR

SEQ ID NO: 36 - protein, 6 aa, Artificial
  thrombin cleavage site
LVPRGS

SEQ ID NO: 37 - DNA, 40 bp, Artificial
  P2A cleavage site
gccacgaagcaagcaggatgttgaagaaaaccccgggcct

SEQ ID NO: 38 - DNA, 54 bp, Artificial
  T2A cleavage site
gagggcagaggaagtcttctaacatgcggtgacgtggaggagaatcccggccct

SEQ ID NO: 39 - DNA, 60 bp, Artificial
  E2A cleavage site
cagtgtactaattatgctctcttgaaattggctggagatgttgagagcaacccaggtccc

SEQ ID NO: 40 - DNA, 1668 bp, Artificial
  firefly luciferase gene
gtgattgcagaattcatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaa

SEQ ID NO: 41 - DNA, 936 bp, Artificial
  renilla luciferase gene
atgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 42 - DNA, 3075 bp, Artificial
  beta - galactosidase gene
atgatagatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggtaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaagcggccgctcgagtctag

SEQ ID NO: 43 - DNA, 1563 bp, Artificial
  human secreted alkaline phosphatase gene
atgattctggggccctgcatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggctatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgatgtgatcctgggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccacggtcatcacgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccggggcggtcccggtccaagcgtctggattga

SEQ ID NO: 44 - DNA, 1524 bp, Artificial
  murine secreted alkaline phosphatase gene
atgtggggtgcctgtctgctattgctgggcttaagtcttcaagtttgccccagtgtcattcctgtggaggaggagaatcctgctttttggaataggaaggcagctgaagccttggatgcagccaagaagctcaagcccattcagacatctgcaaagaatcttgtcatcctcatgggtgatggaatgggtgtctccactgtaacagccaccaggattctgaagggccagcaacaaggtcatctaggcccagagacccagttggcaatggacaggttccctcacatggccctttccaagacttacaacactgacaagcagattcctgactctgctgggacaggcacagcattcttgtgtggagtaaaaaccaacatgaaagtcattggtctttcagctgctgccagattcaaccagtgcaacaccacatggggcaatgaagtggtctctgtaatgcacagggccaaaaaagctgggaaaagtgtgggtgtggtgacaaccacctctgtccagcatgcctctcctgctggaacttatgcccacacagtgaacagaggttggtactctgatgctcagatgcctgcctcagctttacaagatggctgcaaggacatcagcacccagctcatctcaaacatggacatagatgtcatcttagggggtgggagaaagttcatgttcccaaaggggactcctgaccaggagtaccccacagacacaaagcaggctggcacaagattagatggtaggaaccttgtgcaagagtggcttgccaagcatcagggagcaaggtatgtctggaacaggagtgagctaatccaggcctctttgaacaggtctgtcactcacctaatggggttatttgagcccaatgacatgaagtatgagatacacagggaccctgcccaggacccctccttagcagaaatgactgaagttgctgtgaggatgttgtccagaaatccaaaagggttctacctctttgttgaggggggaaggattgatcatggtcaccatgagacagttgcttacagagccttaactgaggctgtgatgtttgattctgctgtggacaaggctgacaaactgacctctgagcaggacacaatgattctagtgactgctgaccacagtcatgttttctcctttgggggctacacccagaggggtgcttcaatctttggcctggcccctttcaaggcagaagatgggaagagtttcacctccatcctctatgggaatggtcctgggtacaagctgcacaatggggccagagctgatgtgacagaagaggagagctccaacccaacctaccagcagcaagcagcagtccctctttcttcagaaacccactctggggaagatgtggccatatttgccagaggcccccaagcccacttggtgcatggagttcaggagcagaattacatagctcatgtaatggcttttgctgcttgcttggagccctacacagactgtggcctagccagcccagcaggccagtcctctgcagtaagcccaggctag

SEQ ID NO: 45 - DNA, 10613 bp, Artificial
  pIM-RAG1-MCS
ggcgcgccagatctgtacattcgaagatatcttaattaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaacggatccgcagcctctttcccacccaccttgggactcagttctgccccagatgaaattcagcacccacatattaaattttcagaatggaaatttaagctgttccgggtgagatcctttgaaaagacacctgaagaagctcaaaaggaaaagaaggattcctttgaggggaaaccctctctggagcaatctccagcagtcctggacaaggctgatggtcagaagccagtcccaactcagccattgttaaaagcccaccctaagttttcgaagaaatttcacgacaacgagaaagcaagaggcaaagcgatccatcaagccaaccttcgacatctctgccgcatctgtgggaattcttttagagctgatgagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttcccgaggaacgtgaccatggagtggcacccccacacaccatcctgtgacatctgcaacactgcccgtcggggactcaagaggaagagtcttcagccaaacttgcagctcagcaaaaaactcaaaactgtgcttgaccaagcaagacaagcccgtcagcacaagagaagagctcaggcaaggatcagcagcaaggatgtcatgaagaagatcgccaactgcagtaagatacatcttagtaccaagctccttgcagtggacttcccagagcactttgtgaaatccatctcctgccagatctgtgaacacattctggctgaccctgtggagaccaactgtaagcatgtcttttgccgggtctgcattctcagatgcctcaaagtcatgggcagctattgtccctcttgccgatatccatgcttccctactgacctggagagtccagtgaagtcctttctgagcgtcttgaattccctgatggtgaaatgtccagcaaaagagtgcaatgaggaggtcagtttggaaaaatataatcaccacatctcaagtcacaaggaatcaaaagagatttttgtgcacattaataaagggggtcgagtaacgcgtgcaggcatgcaagctggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgtaagtgatatctactagatttatcaaaaagagtgttgacttgtgagcgctcacaattgatacttagattcatcgagagggacacgtcgactactaaccttcttctctttcctacagctgagatcaccggcgaaggagggccaccatggcttcttaccctggacaccagcatgcttctgcctttgaccaggctgccagatccaggggccactccaacaggagaactgccctaagacccagaagacagcaggaagccactgaggtgaggcctgagcagaagatgccaaccctgctgagggtgtacattgatggacctcatggcatgggcaagaccaccaccactcaactgctggtggcactgggctccagggatgacattgtgtatgtgcctgagccaatgacctactggagagtgctaggagcctctgagaccattgccaacatctacaccacccagcacaggctggaccagggagaaatctctgctggagatgctgctgtggtgatgacctctgcccagatcacaatgggaatgccctatgctgtgactgatgctgttctggctcctcacattggaggagaggctggctcttctcatgcccctccacctgccctgaccctgatctttgacagacaccccattgcagccctgctgtgctacccagcagcaaggtacctcatgggctccatgaccccacaggctgtgctggcttttgtggccctgatccctccaaccctccctggcaccaacattgttctgggagcactgcctgaagacagacacattgacaggctggcaaagaggcagagacctggagagagactggacctggccatgctggctgcaatcagaagggtgtatggactgctggcaaacactgtgagatacctccagtgtggaggctcttggagagaggactggggacagctctctggaacagcagtgccccctcaaggagctgagccccagtccaatgctggtccaagaccccacattggggacaccctgttcaccctgttcagagcccctgagctgctggctcccaatggagacctgtacaatgtgtttgcctgggctctggatgttctagccaagaggctgaggtccatgcatgtgttcatcctggactatgaccagtcccctgctggatgcagagatgctctgctgcaactaacctctggcatggtgcagacccatgtgaccacccctggcagcatccccaccatctgtgacctagccagaacctttgccagggagatgggagaggccaactaaacctgagctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatccatttttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgacccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccaatattaaacttgatgagctctagagatggtcatgcattttaaaaagaattactcaaaatattgtcttggaataccagagagcaagtgctttaagtataggctgggaagtaaaatgctaaaggaatgagaaggcatttggggttgagttcaacctaagaggcaggggagccacagggaaagacctagcacctgccacagaagagaattaggaagcagaattgaactataagcaattttgaggtgttcgttgggctgcagttgaaatattttttgaggttaatgagacatttgaaatggccgtgtattgtttaactcttgcatagtcctgcatagggaacaatctaataggatttctctgtgaatcaagtcttagaaatttgcttttaatttttatgaaaaacgcccatttctttgtttttgagacagagtcctgctctgtcatccaggctgggttgcagtggcgtgatcttggcccactgcaatctctgcctcctgggttcaggcaattttcctgtctcagcctcccgagtagctgggatttcaagtgcctgccaccatgcccggctaaatttttttgtatttttggtacagatggagtatcaccatgttggccaggctggtctcgaactcctgacctcaagtgattcaccagccttgacctcccaaagtgttgggatcacaggcatgagccactgtgcctgtgccccaaaacaccaatttctgatgtgtgatgcatgtaagatagaacaaacttcagtaaagcggggacttgaaaagaggctttggtaacagctgtcagcattaacccttgcccctccgtacctcctaatcccacccctgctcaaagtatgttcatctgagaatttgtctccataactatgtgactataaaaattctcatcgattttgttagttgatcaattgagggaaaaacatatgttacttgatataactggtgggtcaaaagaattaacccaggcaaatttgagataggtggatgggatgatggattgaaaatacagctgctctctttccaatcatgtactaagtaatttgggaaagattgatctaattgggtctagagagtacacttcacatggcattgtttgactttttttctgcatcgctagcgatctgtgcattacaactcaaatcagtcgggtttcctggcatatgtaattgccaatgttttttaccagaagagaaacattactcccacctcttcttattatgttacaaactatagtgctaatgaccatcgaccaacagtgactttcaggatgacctgtgtgagttttatctgaaaccatgtgaatttttcatcttaaaagtcccttagaatctcagtctatgtacactcaggtttgttgcaggtttagagttccgtgttttttgtttctaatgtagacacagccttataatttacaacagcattcactaattaaaattgtaagcataattactatccacgatacttattattagtttgcattcataaagctcaaaattcacttcatcctttcaagtagtgaataattagtttctttgggtttgcagctttatcatccttttatgacccatttggaagaaataaacaaccaaccccctggaagactgctttaaaaagctggaaatacattgtccagctagtacaatgaggctaatacaatgtggaaaatattacttttctttgattttagtagcctgtttatctttacatttactgaacaaataactattgagcacctaatgtatactgggacccttggggaggcaaagatgaatcaaagattctgtccttaaagaccttaagacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgattaattaacaggactgaccgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacaggatccactagcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcctta

SEQ ID NO: 46 - DNA, 12415 bp, Artificial
  pIM-RAG1-Luc
atggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaataattctagagtcggggcggccggccgcttcgagcagacatgataagatacattgatgagtttggacaaaccacaactagaatgcagtgaaaaaaatgctttatttgtgaaatttgtgatgctattgctttatttgtaaccattaaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaacggatccgcagcctctttcccacccaccttgggactcagttctgccccagatgaaattcagcacccacatattaaattttcagaatggaaatttaagctgttccgggtgagatcctttgaaaagacacctgaagaagctcaaaaggaaaagaaggattcctttgaggggaaaccctctctggagcaatctccagcagtcctggacaaggctgatggtcagaagccagtcccaactcagccattgttaaaagcccaccctaagttttcgaagaaatttcacgacaacgagaaagcaagaggcaaagcgatccatcaagccaaccttcgacatctctgccgcatctgtgggaattcttttagagctgatgagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttcccgaggaacgtgaccatggagtggcacccccacacaccatcctgtgacatctgcaacactgcccgtcggggactcaagaggaagagtcttcagccaaacttgcagctcagcaaaaaactcaaaactgtgcttgaccaagcaagacaagcccgtcagcacaagagaagagctcaggcaaggatcagcagcaaggatgtcatgaagaagatcgccaactgcagtaagatacatcttagtaccaagctccttgcagtggacttcccagagcactttgtgaaatccatctcctgccagatctgtgaacacattctggctgaccctgtggagaccaactgtaagcatgtcttttgccgggtctgcattctcagatgcctcaaagtcatgggcagctattgtccctcttgccgatatccatgcttccctactgacctggagagtccagtgaagtcctttctgagcgtcttgaattccctgatggtgaaatgtccagcaaaagagtgcaatgaggaggtcagtttggaaaaatataatcaccacatctcaagtcacaaggaatcaaaagagatttttgtgcacattaataaagggggtcgagtaacgcgtgcaggcatgcaagctggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgtaagtgatatctactagatttatcaaaaagagtgttgacttgtgagcgctcacaattgatacttagattcatcgagagggacacgtcgactactaaccttcttctctttcctacagctgagatcaccggcgaaggagggccaccatggcttcttaccctggacaccagcatgcttctgcctttgaccaggctgccagatccaggggccactccaacaggagaactgccctaagacccagaagacagcaggaagccactgaggtgaggcctgagcagaagatgccaaccctgctgagggtgtacattgatggacctcatggcatgggcaagaccaccaccactcaactgctggtggcactgggctccagggatgacattgtgtatgtgcctgagccaatgacctactggagagtgctaggagcctctgagaccattgccaacatctacaccacccagcacaggctggaccagggagaaatctctgctggagatgctgctgtggtgatgacctctgcccagatcacaatgggaatgccctatgctgtgactgatgctgttctggctcctcacattggaggagaggctggctcttctcatgcccctccacctgccctgaccctgatctttgacagacaccccattgcagccctgctgtgctacccagcagcaaggtacctcatgggctccatgaccccacaggctgtgctggcttttgtggccctgatccctccaaccctccctggcaccaacattgttctgggagcactgcctgaagacagacacattgacaggctggcaaagaggcagagacctggagagagactggacctggccatgctggctgcaatcagaagggtgtatggactgctggcaaacactgtgagatacctccagtgtggaggctcttggagagaggactggggacagctctctggaacagcagtgccccctcaaggagctgagccccagtccaatgctggtccaagaccccacattggggacaccctgttcaccctgttcagagcccctgagctgctggctcccaatggagacctgtacaatgtgtttgcctgggctctggatgttctagccaagaggctgaggtccatgcatgtgttcatcctggactatgaccagtcccctgctggatgcagagatgctctgctgcaactaacctctggcatggtgcagacccatgtgaccacccctggcagcatccccaccatctgtgacctagccagaacctttgccagggagatgggagaggccaactaaacctgagctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatccatttttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgacccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccaatattaaacttgatgagctctagagatggtcatgcattttaaaaagaattactcaaaatattgtcttggaataccagagagcaagtgctttaagtataggctgggaagtaaaatgctaaaggaatgagaaggcatttggggttgagttcaacctaagaggcaggggagccacagggaaagacctagcacctgccacagaagagaattaggaagcagaattgaactataagcaattttgaggtgttcgttgggctgcagttgaaatattttttgaggttaatgagacatttgaaatggccgtgtattgtttaactcttgcatagtcctgcatagggaacaatctaataggatttctctgtgaatcaagtcttagaaatttgcttttaatttttatgaaaaacgcccatttctttgtttttgagacagagtcctgctctgtcatccaggctgggttgcagtggcgtgatcttggcccactgcaatctctgcctcctgggttcaggcaattttcctgtctcagcctcccgagtagctgggatttcaagtgcctgccaccatgcccggctaaatttttttgtatttttggtacagatggagtatcaccatgttggccaggctggtctcgaactcctgacctcaagtgattcaccagccttgacctcccaaagtgttgggatcacaggcatgagccactgtgcctgtgccccaaaacaccaatttctgatgtgtgatgcatgtaagatagaacaaacttcagtaaagcggggacttgaaaagaggctttggtaacagctgtcagcattaacccttgcccctccgtacctcctaatcccacccctgctcaaagtatgttcatctgagaatttgtctccataactatgtgactataaaaattctcatcgattttgttagttgatcaattgagggaaaaacatatgttacttgatataactggtgggtcaaaagaattaacccaggcaaatttgagataggtggatgggatgatggattgaaaatacagctgctctctttccaatcatgtactaagtaatttgggaaagattgatctaattgggtctagagagtacacttcacatggcattgtttgactttttttctgcatcgctagcgatctgtgcattacaactcaaatcagtcgggtttcctggcatatgtaattgccaatgttttttaccagaagagaaacattactcccacctcttcttattatgttacaaactatagtgctaatgaccatcgaccaacagtgactttcaggatgacctgtgtgagttttatctgaaaccatgtgaatttttcatcttaaaagtcccttagaatctcagtctatgtacactcaggtttgttgcaggtttagagttccgtgttttttgtttctaatgtagacacagccttataatttacaacagcattcactaattaaaattgtaagcataattactatccacgatacttattattagtttgcattcataaagctcaaaattcacttcatcctttcaagtagtgaataattagtttctttgggtttgcagctttatcatccttttatgacccatttggaagaaataaacaaccaaccccctggaagactgctttaaaaagctggaaatacattgtccagctagtacaatgaggctaatacaatgtggaaaatattacttttctttgattttagtagcctgtttatctttacatttactgaacaaataactattgagcacctaatgtatactgggacccttggggaggcaaagatgaatcaaagattctgtccttaaagaccttaagacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgattaattaacaggactgaccgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacaggatccactagcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagccttaggcgcgccagatctgtacattcgaagatatcttaattaagcggccgcgaattcactagtgattgcagaattc

SEQ ID NO: 47 - DNA, 22 bp, Artificial
  F_HS1_PCRSC primer
ggaggattgggaagacaatagc

SEQ ID NO: 48 - DNA, 24 bp, Artificial
  R_HS1_PCRSC primer
ctttcacagtcctgtacatcttgt

SEQ ID NO: 49 - DNA, 717 bp, Artificial
  TagGFP2
atgagcgggggcgaggagctgttcgccggcatcgtgcccgtgctgatcgagctggacggcgacgtgcacggccacaagttcagcgtgcgcggcgagggcgagggcgacgccgactacggcaagctggagatcaagttcatctgcaccaccggcaagctgcccgtgccctggcccaccctggtgaccaccctctgctacggcatccagtgcttcgcccgctaccccgagcacatgaagatgaacgacttcttcaagagcgccatgcccgagggctacatccaggagcgcaccatccagttccaggacgacggcaagtacaagacccgcggcgaggtgaagttcgagggcgacaccctggtgaaccgcatcgagctgaagggcaaggacttcaaggaggacggcaacatcctgggccacaagctggagtacagcttcaacagccacaacgtgtacatccgccccgacaaggccaacaacggcctggaggctaacttcaagacccgccacaacatcgagggcggcggcgtgcagctggccgaccactaccagaccaacgtgcccctgggcgacggccccgtgctgatccccatcaaccactacctgagcactcagaccaagatcagcaaggaccgcaacgaggcccgcgaccacatggtgctcctggagtccttcagcgcctgctgccacacccacggcatggacgagctgtacaggtaa

SEQ ID NO: 50 - DNA, 11805 bp, Artificial
  pIM-RAG1-EF1-MCS
gtttaaacaatattcctgcagggcagcccggggagcggccgcggagctccaggaattctgcagatcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatccgcagcctctttcccacccaccttgggactcagttctgccccagatgaaattcagcacccacatattaaattttcagaatggaaatttaagctgttccgggtgagatcctttgaaaagacacctgaagaagctcaaaaggaaaagaaggattcctttgaggggaaaccctctctggagcaatctccagcagtcctggacaaggctgatggtcagaagccagtcccaactcagccattgttaaaagcccaccctaagttttcgaagaaatttcacgacaacgagaaagcaagaggcaaagcgatccatcaagccaaccttcgacatctctgccgcatctgtgggaattcttttagagctgatgagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttcccgaggaacgtgaccatggagtggcacccccacacaccatcctgtgacatctgcaacactgcccgtcggggactcaagaggaagagtcttcagccaaacttgcagctcagcaaaaaactcaaaactgtgcttgaccaagcaagacaagcccgtcagcacaagagaagagctcaggcaaggatcagcagcaaggatgtcatgaagaagatcgccaactgcagtaagatacatcttagtaccaagctccttgcagtggacttcccagagcactttgtgaaatccatctcctgccagatctgtgaacacattctggctgaccctgtggagaccaactgtaagcatgtcttttgccgggtctgcattctcagatgcctcaaagtcatgggcagctattgtccctcttgccgatatccatgcttccctactgacctggagagtccagtgaagtcctttctgagcgtcttgaattccctgatggtgaaatgtccagcaaaagagtgcaatgaggaggtcagtttggaaaaatataatcaccacatctcaagtcacaaggaatcaaaagagatttttgtgcacattaataaagggggtcgagtaacgcgtgcaggcatgcaagctggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgtaagtgatatctactagatttatcaaaaagagtgttgacttgtgagcgctcacaattgatacttagattcatcgagagggacacgtcgactactaaccttcttctctttcctacagctgagatcaccggcgaaggagggccaccatggcttcttaccctggacaccagcatgcttctgcctttgaccaggctgccagatccaggggccactccaacaggagaactgccctaagacccagaagacagcaggaagccactgaggtgaggcctgagcagaagatgccaaccctgctgagggtgtacattgatggacctcatggcatgggcaagaccaccaccactcaactgctggtggcactgggctccagggatgacattgtgtatgtgcctgagccaatgacctactggagagtgctaggagcctctgagaccattgccaacatctacaccacccagcacaggctggaccagggagaaatctctgctggagatgctgctgtggtgatgacctctgcccagatcacaatgggaatgccctatgctgtgactgatgctgttctggctcctcacattggaggagaggctggctcttctcatgcccctccacctgccctgaccctgatctttgacagacaccccattgcagccctgctgtgctacccagcagcaaggtacctcatgggctccatgaccccacaggctgtgctggcttttgtggccctgatccctccaaccctccctggcaccaacattgttctgggagcactgcctgaagacagacacattgacaggctggcaaagaggcagagacctggagagagactggacctggccatgctggctgcaatcagaagggtgtatggactgctggcaaacactgtgagatacctccagtgtggaggctcttggagagaggactggggacagctctctggaacagcagtgccccctcaaggagctgagccccagtccaatgctggtccaagaccccacattggggacaccctgttcaccctgttcagagcccctgagctgctggctcccaatggagacctgtacaatgtgtttgcctgggctctggatgttctagccaagaggctgaggtccatgcatgtgttcatcctggactatgaccagtcccctgctggatgcagagatgctctgctgcaactaacctctggcatggtgcagacccatgtgaccacccctggcagcatccccaccatctgtgacctagccagaacctttgccagggagatgggagaggccaactaaacctgagctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatcatttagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccaatattaaacttgatgagctctagagatggtcatgcattttaaaaagaattactcaaaatattgtcttggaataccagagagcaagtgctttaagtataggctgggaagtaaaatgctaaaggaatgagaaggcatttggggttgagttcaacctaagaggcaggggagccacagggaaagacctagcacctgccacagaagagaattaggaagcagaattgaactataagcaattttgaggtgttcgttgggctgcagttgaaatattttttgaggttaatgagacatttgaaatggccgtgtattgtttaactcttgcatagtcctgcatagggaacaatctaataggatttctctgtgaatcaagtcttagaaatttgcttttaatttttatgaaaaacgcccatttctttgtttttgagacagagtcctgctctgtcatccaggctgggttgcagtggcgtgatcttggcccactgcaatctctgcctcctgggttcaggcaattttcctgtctcagcctcccgagtagctgggatttcaagtgcctgccaccatgcccggctaaatttttttgtatttttggtacagatggagtatcaccatgttggccaggctggtctcgaactcctgacctcaagtgattcaccagccttgacctcccaaagtgttgggatcacaggcatgagccactgtgcctgtgccccaaaacaccaatttctgatgtgtgatgcatgtaagatagaacaaacttcagtaaagcggggacttgaaaagaggctttggtaacagctgtcagcattaacccttgcccctccgtacctcctaatcccacccctgctcaaagtatgttcatctgagaatttgtctccataactatgtgactataaaaattctcatcgattttgttagttgatcaattgagggaaaaacatatgttacttgatataactggtgggtcaaaagaattaacccaggcaaatttgagataggtggatgggatgatggattgaaaatacagctgctctctttccaatcatgtactaagtaatttgggaaagattgatctaattgggtctagagagtacacttcacatggcattgtttgactttttttctgcatcgctagcgatctgtgcattacaactcaaatcagtcgggtttcctggcatatgtaattgccaatgttttttaccagaagagaaacattactcccacctcttcttattatgttacaaactatagtgctaatgaccatcgaccaacagtgactttcaggatgacctgtgtgagttttatctgaaaccatgtgaatttttcatcttaaaagtcccttagaatctcagtctatgtacactcaggtttgttgcaggtttagagttccgtgttttttgtttctaatgtagacacagccttataatttacaacagcattcactaattaaaattgtaagcataattactatccacgatacttattattagtttgcattcataaagctcaaaattcacttcatcctttcaagtagtgaataattagtttctttgggtttgcagctttatcatccttttatgacccatttggaagaaataaacaaccaaccccctggaagactgctttaaaaagctggaaatacattgtccagctagtacaatgaggctaatacaatgtggaaaatattacttttctttgattttagtagcctgtttatctttacatttactgaacaaataactattgagcacctaatgtatactgggacccttggggaggcaaagatgaatcaaagattctgtccttaaagaccttaagttaagacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgattaattaacaggactgaccgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacaggatccactagtaacggccgccagtgtgctggaattccgaggtcgacggtatcgataagggcgcgccaaagctaactgtaggactgagtctattctaaactgaaagcctggacatctggagtaccagggggagatgacgtgttacgggcttccataaaagcagctggctttgaatggaaggagccaagaggccagcacaggagcggattcgtcgctttcacggccatcgagccgaacctctcgcaagtccgtgagccgttaaggaggcccccagtcccgacccttcgccccaagcccctcggggtccccgggcctggtactccttgccacacgggaggggcgcggaagccggggcggaggaggagccaaccccgggctgggctgagacccgcagaggaagacgctctagggatttgtcccggactagcgagatggcaaggctgaggacgggaggctgattgagaggcgaaggtacaccctaatctcaatacaacctttggagctaagccagcaatggtagagggaagattctgcacgtcccttccaggcggcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgg

SEQ ID NO: 51 - DNA, 10688 bp, Artificial
  pIM-RAG1-GAS5-MCS
gtttaaacaatattcctgcagggcagcccggggagcggccgcggagctccaggaattctgcagatcactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatccgcagcctctttcccacccaccttgggactcagttctgccccagatgaaattcagcacccacatattaaattttcagaatggaaatttaagctgttccgggtgagatcctttgaaaagacacctgaagaagctcaaaaggaaaagaaggattcctttgaggggaaaccctctctggagcaatctccagcagtcctggacaaggctgatggtcagaagccagtcccaactcagccattgttaaaagcccaccctaagttttcgaagaaatttcacgacaacgagaaagcaagaggcaaagcgatccatcaagccaaccttcgacatctctgccgcatctgtgggaattcttttagagctgatgagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttcccgaggaacgtgaccatggagtggcacccccacacaccatcctgtgacatctgcaacactgcccgtcggggactcaagaggaagagtcttcagccaaacttgcagctcagcaaaaaactcaaaactgtgcttgaccaagcaagacaagcccgtcagcacaagagaagagctcaggcaaggatcagcagcaaggatgtcatgaagaagatcgccaactgcagtaagatacatcttagtaccaagctccttgcagtggacttcccagagcactttgtgaaatccatctcctgccagatctgtgaacacattctggctgaccctgtggagaccaactgtaagcatgtcttttgccgggtctgcattctcagatgcctcaaagtcatgggcagctattgtccctcttgccgatatccatgcttccctactgacctggagagtccagtgaagtcctttctgagcgtcttgaattccctgatggtgaaatgtccagcaaaagagtgcaatgaggaggtcagtttggaaaaatataatcaccacatctcaagtcacaaggaatcaaaagagatttttgtgcacattaataaagggggtcgagtaacgcgtgcaggcatgcaagctggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgtaagtgatatctactagatttatcaaaaagagtgttgacttgtgagcgctcacaattgatacttagattcatcgagagggacacgtcgactactaaccttcttctctttcctacagctgagatcaccggcgaaggagggccaccatggcttcttaccctggacaccagcatgcttctgcctttgaccaggctgccagatccaggggccactccaacaggagaactgccctaagacccagaagacagcaggaagccactgaggtgaggcctgagcagaagatgccaaccctgctgagggtgtacattgatggacctcatggcatgggcaagaccaccaccactcaactgctggtggcactgggctccagggatgacattgtgtatgtgcctgagccaatgacctactggagagtgctaggagcctctgagaccattgccaacatctacaccacccagcacaggctggaccagggagaaatctctgctggagatgctgctgtggtgatgacctctgcccagatcacaatgggaatgccctatgctgtgactgatgctgttctggctcctcacattggaggagaggctggctcttctcatgcccctccacctgccctgaccctgatctttgacagacaccccattgcagccctgctgtgctacccagcagcaaggtacctcatgggctccatgaccccacaggctgtgctggcttttgtggccctgatccctccaaccctccctggcaccaacattgttctgggagcactgcctgaagacagacacattgacaggctggcaaagaggcagagacctggagagagactggacctggccatgctggctgcaatcagaagggtgtatggactgctggcaaacactgtgagatacctccagtgtggaggctcttggagagaggactggggacagctctctggaacagcagtgccccctcaaggagctgagccccagtccaatgctggtccaagaccccacattggggacaccctgttcaccctgttcagagcccctgagctgctggctcccaatggagacctgtacaatgtgtttgcctgggctctggatgttctagccaagaggctgaggtccatgcatgtgttcatcctggactatgaccagtcccctgctggatgcagagatgctctgctgcaactaacctctggcatggtgcagacccatgtgaccacccctggcagcatccccaccatctgtgacctagccagaacctttgccagggagatgggagaggccaactaaacctgagctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatcatttagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccaatattaaacttgatgagctctagagatggtcatgcattttaaaaagaattactcaaaatattgtcttggaataccagagagcaagtgctttaagtataggctgggaagtaaaatgctaaaggaatgagaaggcatttggggttgagttcaacctaagaggcaggggagccacagggaaagacctagcacctgccacagaagagaattaggaagcagaattgaactataagcaattttgaggtgttcgttgggctgcagttgaaatattttttgaggttaatgagacatttgaaatggccgtgtattgtttaactcttgcatagtcctgcatagggaacaatctaataggatttctctgtgaatcaagtcttagaaatttgcttttaatttttatgaaaaacgcccatttctttgtttttgagacagagtcctgctctgtcatccaggctgggttgcagtggcgtgatcttggcccactgcaatctctgcctcctgggttcaggcaattttcctgtctcagcctcccgagtagctgggatttcaagtgcctgccaccatgcccggctaaatttttttgtatttttggtacagatggagtatcaccatgttggccaggctggtctcgaactcctgacctcaagtgattcaccagccttgacctcccaaagtgttgggatcacaggcatgagccactgtgcctgtgccccaaaacaccaatttctgatgtgtgatgcatgtaagatagaacaaacttcagtaaagcggggacttgaaaagaggctttggtaacagctgtcagcattaacccttgcccctccgtacctcctaatcccacccctgctcaaagtatgttcatctgagaatttgtctccataactatgtgactataaaaattctcatcgattttgttagttgatcaattgagggaaaaacatatgttacttgatataactggtgggtcaaaagaattaacccaggcaaatttgagataggtggatgggatgatggattgaaaatacagctgctctctttccaatcatgtactaagtaatttgggaaagattgatctaattgggtctagagagtacacttcacatggcattgtttgactttttttctgcatcgctagcgatctgtgcattacaactcaaatcagtcgggtttcctggcatatgtaattgccaatgttttttaccagaagagaaacattactcccacctcttcttattatgttacaaactatagtgctaatgaccatcgaccaacagtgactttcaggatgacctgtgtgagttttatctgaaaccatgtgaatttttcatcttaaaagtcccttagaatctcagtctatgtacactcaggtttgttgcaggtttagagttccgtgttttttgtttctaatgtagacacagccttataatttacaacagcattcactaattaaaattgtaagcataattactatccacgatacttattattagtttgcattcataaagctcaaaattcacttcatcctttcaagtagtgaataattagtttctttgggtttgcagctttatcatccttttatgacccatttggaagaaataaacaaccaaccccctggaagactgctttaaaaagctggaaatacattgtccagctagtacaatgaggctaatacaatgtggaaaatattacttttctttgattttagtagcctgtttatctttacatttactgaacaaataactattgagcacctaatgtatactgggacccttggggaggcaaagatgaatcaaagattctgtccttaaagaccttaagttaagacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgattaattaacaggactgaccgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacaggatccactagtaacggccgccagtgtgctggaattccgaggtcgacggtatcgataagggcgcgcctgcggcaagggagttgcgggcgcacaggtaaggcgcgggaccgggaagggggcggcgccccgggatcggtctagggcggcggaggctgccggggcgggggtgtgtctgggtgtggccccggcgcgcgcggggtcctgagggaggggctcgccaagggggcgggcccgcgagctcgacagccccgagccaaaggggcggaaggtcgccgagtgctgggaggggaggtggggcagcgactgagcgccgcaggagctggctgcaacccagacgcggcccgggagcgtcgggtatgagctaacggggcgggggtgcggggaacgagcccagtcaaggagcagcacctacctccgggggtggaaatgggccggaatgggccggaacaccgtcccggaagtgaaaccccccgccccctcctccgcagcgagcgacgtgccggaaggaaatcagtcaccctccccccgcccctccccccagcactttccttgcaggagccccctcccctcagcctgtactcctcagggaggcggagggcgggcctatcgtgcccgcggcaactccgccctccgggtctcgggggcgtggccagagggaaagttttgtgggcctgaagaaggtggggcttgaggaggagtctgagggcgtgtgaggggcggggtttaagtggacgcagcaagcagctttgcgtcttgacgtcacgaagaccttatatactcgactcagccgccatctcagcctttcggagctgtgcggt

SEQ ID NO: 52 - DNA, 11328 bp, Artificial
  pIM-RAG1-TagGFP2
atgagcgggggcgaggagctgttcgccggcatcgtgcccgtgctgatcgagctggacggcgacgtgcacggccacaagttcagcgtgcgcggcgagggcgagggcgacgccgactacggcaagctggagatcaagttcatctgcaccaccggcaagctgcccgtgccctggcccaccctggtgaccaccctctgctacggcatccagtgcttcgcccgctaccccgagcacatgaagatgaacgacttcttcaagagcgccatgcccgagggctacatccaggagcgcaccatccagttccaggacgacggcaagtacaagacccgcggcgaggtgaagttcgagggcgacaccctggtgaaccgcatcgagctgaagggcaaggacttcaaggaggacggcaacatcctgggccacaagctggagtacagcttcaacagccacaacgtgtacatccgccccgacaaggccaacaacggcctggaggctaacttcaagacccgccacaacatcgagggcggcggcgtgcagctggccgaccactaccagaccaacgtgcccctgggcgacggccccgtgctgatccccatcaaccactacctgagcactcagaccaagatcagcaaggaccgcaacgaggcccgcgaccacatggtgctcctggagtccttcagcgcctgctgccacacccacggcatggacgagctgtacaggtaacccggggagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaacggatccgcagcctctttcccacccaccttgggactcagttctgccccagatgaaattcagcacccacatattaaattttcagaatggaaatttaagctgttccgggtgagatcctttgaaaagacacctgaagaagctcaaaaggaaaagaaggattcctttgaggggaaaccctctctggagcaatctccagcagtcctggacaaggctgatggtcagaagccagtcccaactcagccattgttaaaagcccaccctaagttttcgaagaaatttcacgacaacgagaaagcaagaggcaaagcgatccatcaagccaaccttcgacatctctgccgcatctgtgggaattcttttagagctgatgagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttcccgaggaacgtgaccatggagtggcacccccacacaccatcctgtgacatctgcaacactgcccgtcggggactcaagaggaagagtcttcagccaaacttgcagctcagcaaaaaactcaaaactgtgcttgaccaagcaagacaagcccgtcagcacaagagaagagctcaggcaaggatcagcagcaaggatgtcatgaagaagatcgccaactgcagtaagatacatcttagtaccaagctccttgcagtggacttcccagagcactttgtgaaatccatctcctgccagatctgtgaacacattctggctgaccctgtggagaccaactgtaagcatgtcttttgccgggtctgcattctcagatgcctcaaagtcatgggcagctattgtccctcttgccgatatccatgcttccctactgacctggagagtccagtgaagtcctttctgagcgtcttgaattccctgatggtgaaatgtccagcaaaagagtgcaatgaggaggtcagtttggaaaaatataatcaccacatctcaagtcacaaggaatcaaaagagatttttgtgcacattaataaagggggtcgagtaacgcgtgcaggcatgcaagctggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgtaagtgatatctactagatttatcaaaaagagtgttgacttgtgagcgctcacaattgatacttagattcatcgagagggacacgtcgactactaaccttcttctctttcctacagctgagatcaccggcgaaggagggccaccatggcttcttaccctggacaccagcatgcttctgcctttgaccaggctgccagatccaggggccactccaacaggagaactgccctaagacccagaagacagcaggaagccactgaggtgaggcctgagcagaagatgccaaccctgctgagggtgtacattgatggacctcatggcatgggcaagaccaccaccactcaactgctggtggcactgggctccagggatgacattgtgtatgtgcctgagccaatgacctactggagagtgctaggagcctctgagaccattgccaacatctacaccacccagcacaggctggaccagggagaaatctctgctggagatgctgctgtggtgatgacctctgcccagatcacaatgggaatgccctatgctgtgactgatgctgttctggctcctcacattggaggagaggctggctcttctcatgcccctccacctgccctgaccctgatctttgacagacaccccattgcagccctgctgtgctacccagcagcaaggtacctcatgggctccatgaccccacaggctgtgctggcttttgtggccctgatccctccaaccctccctggcaccaacattgttctgggagcactgcctgaagacagacacattgacaggctggcaaagaggcagagacctggagagagactggacctggccatgctggctgcaatcagaagggtgtatggactgctggcaaacactgtgagatacctccagtgtggaggctcttggagagaggactggggacagctctctggaacagcagtgccccctcaaggagctgagccccagtccaatgctggtccaagaccccacattggggacaccctgttcaccctgttcagagcccctgagctgctggctcccaatggagacctgtacaatgtgtttgcctgggctctggatgttctagccaagaggctgaggtccatgcatgtgttcatcctggactatgaccagtcccctgctggatgcagagatgctctgctgcaactaacctctggcatggtgcagacccatgtgaccacccctggcagcatccccaccatctgtgacctagccagaacctttgccagggagatgggagaggccaactaaacctgagctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatccatttttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgacccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccaatattaaacttgatgagctctagagatggtcatgcattttaaaaagaattactcaaaatattgtcttggaataccagagagcaagtgctttaagtataggctgggaagtaaaatgctaaaggaatgagaaggcatttggggttgagttcaacctaagaggcaggggagccacagggaaagacctagcacctgccacagaagagaattaggaagcagaattgaactataagcaattttgaggtgttcgttgggctgcagttgaaatattttttgaggttaatgagacatttgaaatggccgtgtattgtttaactcttgcatagtcctgcatagggaacaatctaataggatttctctgtgaatcaagtcttagaaatttgcttttaatttttatgaaaaacgcccatttctttgtttttgagacagagtcctgctctgtcatccaggctgggttgcagtggcgtgatcttggcccactgcaatctctgcctcctgggttcaggcaattttcctgtctcagcctcccgagtagctgggatttcaagtgcctgccaccatgcccggctaaatttttttgtatttttggtacagatggagtatcaccatgttggccaggctggtctcgaactcctgacctcaagtgattcaccagccttgacctcccaaagtgttgggatcacaggcatgagccactgtgcctgtgccccaaaacaccaatttctgatgtgtgatgcatgtaagatagaacaaacttcagtaaagcggggacttgaaaagaggctttggtaacagctgtcagcattaacccttgcccctccgtacctcctaatcccacccctgctcaaagtatgttcatctgagaatttgtctccataactatgtgactataaaaattctcatcgattttgttagttgatcaattgagggaaaaacatatgttacttgatataactggtgggtcaaaagaattaacccaggcaaatttgagataggtggatgggatgatggattgaaaatacagctgctctctttccaatcatgtactaagtaatttgggaaagattgatctaattgggtctagagagtacacttcacatggcattgtttgactttttttctgcatcgctagcgatctgtgcattacaactcaaatcagtcgggtttcctggcatatgtaattgccaatgttttttaccagaagagaaacattactcccacctcttcttattatgttacaaactatagtgctaatgaccatcgaccaacagtgactttcaggatgacctgtgtgagttttatctgaaaccatgtgaatttttcatcttaaaagtcccttagaatctcagtctatgtacactcaggtttgttgcaggtttagagttccgtgttttttgtttctaatgtagacacagccttataatttacaacagcattcactaattaaaattgtaagcataattactatccacgatacttattattagtttgcattcataaagctcaaaattcacttcatcctttcaagtagtgaataattagtttctttgggtttgcagctttatcatccttttatgacccatttggaagaaataaacaaccaaccccctggaagactgctttaaaaagctggaaatacattgtccagctagtacaatgaggctaatacaatgtggaaaatattacttttctttgattttagtagcctgtttatctttacatttactgaacaaataactattgagcacctaatgtatactgggacccttggggaggcaaagatgaatcaaagattctgtccttaaagaccttaagacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgattaattaacaggactgaccgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacaggatccactagcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagccttaggcgcgcctcgcgagtttaaaccgccacc

SEQ ID NO: 53 - DNA, 12507 bp, Artificial
  pIM-RAG1-EF1-TagGFP
atgagcgggggcgaggagctgttcgccggcatcgtgcccgtgctgatcgagctggacggcgacgtgcacggccacaagttcagcgtgcgcggcgagggcgagggcgacgccgactacggcaagctggagatcaagttcatctgcaccaccggcaagctgcccgtgccctggcccaccctggtgaccaccctctgctacggcatccagtgcttcgcccgctaccccgagcacatgaagatgaacgacttcttcaagagcgccatgcccgagggctacatccaggagcgcaccatccagttccaggacgacggcaagtacaagacccgcggcgaggtgaagttcgagggcgacaccctggtgaaccgcatcgagctgaagggcaaggacttcaaggaggacggcaacatcctgggccacaagctggagtacagcttcaacagccacaacgtgtacatccgccccgacaaggccaacaacggcctggaggctaacttcaagacccgccacaacatcgagggcggcggcgtgcagctggccgaccactaccagaccaacgtgcccctgggcgacggccccgtgctgatccccatcaaccactacctgagcactcagaccaagatcagcaaggaccgcaacgaggcccgcgaccacatggtgctcctggagtccttcagcgcctgctgccacacccacggcatggacgagctgtacaggtaacccggggagcggccgcggagctccaggaattctgcagatcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatccgcagcctctttcccacccaccttgggactcagttctgccccagatgaaattcagcacccacatattaaattttcagaatggaaatttaagctgttccgggtgagatcctttgaaaagacacctgaagaagctcaaaaggaaaagaaggattcctttgaggggaaaccctctctggagcaatctccagcagtcctggacaaggctgatggtcagaagccagtcccaactcagccattgttaaaagcccaccctaagttttcgaagaaatttcacgacaacgagaaagcaagaggcaaagcgatccatcaagccaaccttcgacatctctgccgcatctgtgggaattcttttagagctgatgagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttcccgaggaacgtgaccatggagtggcacccccacacaccatcctgtgacatctgcaacactgcccgtcggggactcaagaggaagagtcttcagccaaacttgcagctcagcaaaaaactcaaaactgtgcttgaccaagcaagacaagcccgtcagcacaagagaagagctcaggcaaggatcagcagcaaggatgtcatgaagaagatcgccaactgcagtaagatacatcttagtaccaagctccttgcagtggacttcccagagcactttgtgaaatccatctcctgccagatctgtgaacacattctggctgaccctgtggagaccaactgtaagcatgtcttttgccgggtctgcattctcagatgcctcaaagtcatgggcagctattgtccctcttgccgatatccatgcttccctactgacctggagagtccagtgaagtcctttctgagcgtcttgaattccctgatggtgaaatgtccagcaaaagagtgcaatgaggaggtcagtttggaaaaatataatcaccacatctcaagtcacaaggaatcaaaagagatttttgtgcacattaataaagggggtcgagtaacgcgtgcaggcatgcaagctggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgtaagtgatatctactagatttatcaaaaagagtgttgacttgtgagcgctcacaattgatacttagattcatcgagagggacacgtcgactactaaccttcttctctttcctacagctgagatcaccggcgaaggagggccaccatggcttcttaccctggacaccagcatgcttctgcctttgaccaggctgccagatccaggggccactccaacaggagaactgccctaagacccagaagacagcaggaagccactgaggtgaggcctgagcagaagatgccaaccctgctgagggtgtacattgatggacctcatggcatgggcaagaccaccaccactcaactgctggtggcactgggctccagggatgacattgtgtatgtgcctgagccaatgacctactggagagtgctaggagcctctgagaccattgccaacatctacaccacccagcacaggctggaccagggagaaatctctgctggagatgctgctgtggtgatgacctctgcccagatcacaatgggaatgccctatgctgtgactgatgctgttctggctcctcacattggaggagaggctggctcttctcatgcccctccacctgccctgaccctgatctttgacagacaccccattgcagccctgctgtgctacccagcagcaaggtacctcatgggctccatgaccccacaggctgtgctggcttttgtggccctgatccctccaaccctccctggcaccaacattgttctgggagcactgcctgaagacagacacattgacaggctggcaaagaggcagagacctggagagagactggacctggccatgctggctgcaatcagaagggtgtatggactgctggcaaacactgtgagatacctccagtgtggaggctcttggagagaggactggggacagctctctggaacagcagtgccccctcaaggagctgagccccagtccaatgctggtccaagaccccacattggggacaccctgttcaccctgttcagagcccctgagctgctggctcccaatggagacctgtacaatgtgtttgcctgggctctggatgttctagccaagaggctgaggtccatgcatgtgttcatcctggactatgaccagtcccctgctggatgcagagatgctctgctgcaactaacctctggcatggtgcagacccatgtgaccacccctggcagcatccccaccatctgtgacctagccagaacctttgccagggagatgggagaggccaactaaacctgagctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatcatttagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccaatattaaacttgatgagctctagagatggtcatgcattttaaaaagaattactcaaaatattgtcttggaataccagagagcaagtgctttaagtataggctgggaagtaaaatgctaaaggaatgagaaggcatttggggttgagttcaacctaagaggcaggggagccacagggaaagacctagcacctgccacagaagagaattaggaagcagaattgaactataagcaattttgaggtgttcgttgggctgcagttgaaatattttttgaggttaatgagacatttgaaatggccgtgtattgtttaactcttgcatagtcctgcatagggaacaatctaataggatttctctgtgaatcaagtcttagaaatttgcttttaatttttatgaaaaacgcccatttctttgtttttgagacagagtcctgctctgtcatccaggctgggttgcagtggcgtgatcttggcccactgcaatctctgcctcctgggttcaggcaattttcctgtctcagcctcccgagtagctgggatttcaagtgcctgccaccatgcccggctaaatttttttgtatttttggtacagatggagtatcaccatgttggccaggctggtctcgaactcctgacctcaagtgattcaccagccttgacctcccaaagtgttgggatcacaggcatgagccactgtgcctgtgccccaaaacaccaatttctgatgtgtgatgcatgtaagatagaacaaacttcagtaaagcggggacttgaaaagaggctttggtaacagctgtcagcattaacccttgcccctccgtacctcctaatcccacccctgctcaaagtatgttcatctgagaatttgtctccataactatgtgactataaaaattctcatcgattttgttagttgatcaattgagggaaaaacatatgttacttgatataactggtgggtcaaaagaattaacccaggcaaatttgagataggtggatgggatgatggattgaaaatacagctgctctctttccaatcatgtactaagtaatttgggaaagattgatctaattgggtctagagagtacacttcacatggcattgtttgactttttttctgcatcgctagcgatctgtgcattacaactcaaatcagtcgggtttcctggcatatgtaattgccaatgttttttaccagaagagaaacattactcccacctcttcttattatgttacaaactatagtgctaatgaccatcgaccaacagtgactttcaggatgacctgtgtgagttttatctgaaaccatgtgaatttttcatcttaaaagtcccttagaatctcagtctatgtacactcaggtttgttgcaggtttagagttccgtgttttttgtttctaatgtagacacagccttataatttacaacagcattcactaattaaaattgtaagcataattactatccacgatacttattattagtttgcattcataaagctcaaaattcacttcatcctttcaagtagtgaataattagtttctttgggtttgcagctttatcatccttttatgacccatttggaagaaataaacaaccaaccccctggaagactgctttaaaaagctggaaatacattgtccagctagtacaatgaggctaatacaatgtggaaaatattacttttctttgattttagtagcctgtttatctttacatttactgaacaaataactattgagcacctaatgtatactgggacccttggggaggcaaagatgaatcaaagattctgtccttaaagaccttaagttaagacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgattaattaacaggactgaccgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacaggatccactagtaacggccgccagtgtgctggaattccgaggtcgacggtatcgataagggcgcgccaaagctaactgtaggactgagtctattctaaactgaaagcctggacatctggagtaccagggggagatgacgtgttacgggcttccataaaagcagctggctttgaatggaaggagccaagaggccagcacaggagcggattcgtcgctttcacggccatcgagccgaacctctcgcaagtccgtgagccgttaaggaggcccccagtcccgacccttcgccccaagcccctcggggtccccgggcctggtactccttgccacacgggaggggcgcggaagccggggcggaggaggagccaaccccgggctgggctgagacccgcagaggaagacgctctagggatttgtcccggactagcgagatggcaaggctgaggacgggaggctgattgagaggcgaaggtacaccctaatctcaatacaacctttggagctaagccagcaatggtagagggaagattctgcacgtcccttccaggcggcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgggtttaaaccgccacc

SEQ ID NO: 54 - DNA, 11395 bp, Artificial
  pIM-RAG1-GAS5-TagGFP2
atgagcgggggcgaggagctgttcgccggcatcgtgcccgtgctgatcgagctggacggcgacgtgcacggccacaagttcagcgtgcgcggcgagggcgagggcgacgccgactacggcaagctggagatcaagttcatctgcaccaccggcaagctgcccgtgccctggcccaccctggtgaccaccctctgctacggcatccagtgcttcgcccgctaccccgagcacatgaagatgaacgacttcttcaagagcgccatgcccgagggctacatccaggagcgcaccatccagttccaggacgacggcaagtacaagacccgcggcgaggtgaagttcgagggcgacaccctggtgaaccgcatcgagctgaagggcaaggacttcaaggaggacggcaacatcctgggccacaagctggagtacagcttcaacagccacaacgtgtacatccgccccgacaaggccaacaacggcctggaggctaacttcaagacccgccacaacatcgagggcggcggcgtgcagctggccgaccactaccagaccaacgtgcccctgggcgacggccccgtgctgatccccatcaaccactacctgagcactcagaccaagatcagcaaggaccgcaacgaggcccgcgaccacatggtgctcctggagtccttcagcgcctgctgccacacccacggcatggacgagctgtacaggtaacccggggagcggccgcggagctccaggaattctgcagatcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatccgcagcctctttcccacccaccttgggactcagttctgccccagatgaaattcagcacccacatattaaattttcagaatggaaatttaagctgttccgggtgagatcctttgaaaagacacctgaagaagctcaaaaggaaaagaaggattcctttgaggggaaaccctctctggagcaatctccagcagtcctggacaaggctgatggtcagaagccagtcccaactcagccattgttaaaagcccaccctaagttttcgaagaaatttcacgacaacgagaaagcaagaggcaaagcgatccatcaagccaaccttcgacatctctgccgcatctgtgggaattcttttagagctgatgagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttcccgaggaacgtgaccatggagtggcacccccacacaccatcctgtgacatctgcaacactgcccgtcggggactcaagaggaagagtcttcagccaaacttgcagctcagcaaaaaactcaaaactgtgcttgaccaagcaagacaagcccgtcagcacaagagaagagctcaggcaaggatcagcagcaaggatgtcatgaagaagatcgccaactgcagtaagatacatcttagtaccaagctccttgcagtggacttcccagagcactttgtgaaatccatctcctgccagatctgtgaacacattctggctgaccctgtggagaccaactgtaagcatgtcttttgccgggtctgcattctcagatgcctcaaagtcatgggcagctattgtccctcttgccgatatccatgcttccctactgacctggagagtccagtgaagtcctttctgagcgtcttgaattccctgatggtgaaatgtccagcaaaagagtgcaatgaggaggtcagtttggaaaaatataatcaccacatctcaagtcacaaggaatcaaaagagatttttgtgcacattaataaagggggtcgagtaacgcgtgcaggcatgcaagctggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgtaagtgatatctactagatttatcaaaaagagtgttgacttgtgagcgctcacaattgatacttagattcatcgagagggacacgtcgactactaaccttcttctctttcctacagctgagatcaccggcgaaggagggccaccatggcttcttaccctggacaccagcatgcttctgcctttgaccaggctgccagatccaggggccactccaacaggagaactgccctaagacccagaagacagcaggaagccactgaggtgaggcctgagcagaagatgccaaccctgctgagggtgtacattgatggacctcatggcatgggcaagaccaccaccactcaactgctggtggcactgggctccagggatgacattgtgtatgtgcctgagccaatgacctactggagagtgctaggagcctctgagaccattgccaacatctacaccacccagcacaggctggaccagggagaaatctctgctggagatgctgctgtggtgatgacctctgcccagatcacaatgggaatgccctatgctgtgactgatgctgttctggctcctcacattggaggagaggctggctcttctcatgcccctccacctgccctgaccctgatctttgacagacaccccattgcagccctgctgtgctacccagcagcaaggtacctcatgggctccatgaccccacaggctgtgctggcttttgtggccctgatccctccaaccctccctggcaccaacattgttctgggagcactgcctgaagacagacacattgacaggctggcaaagaggcagagacctggagagagactggacctggccatgctggctgcaatcagaagggtgtatggactgctggcaaacactgtgagatacctccagtgtggaggctcttggagagaggactggggacagctctctggaacagcagtgccccctcaaggagctgagccccagtccaatgctggtccaagaccccacattggggacaccctgttcaccctgttcagagcccctgagctgctggctcccaatggagacctgtacaatgtgtttgcctgggctctggatgttctagccaagaggctgaggtccatgcatgtgttcatcctggactatgaccagtcccctgctggatgcagagatgctctgctgcaactaacctctggcatggtgcagacccatgtgaccacccctggcagcatccccaccatctgtgacctagccagaacctttgccagggagatgggagaggccaactaaacctgagctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatcatttagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccaatattaaacttgatgagctctagagatggtcatgcattttaaaaagaattactcaaaatattgtcttggaataccagagagcaagtgctttaagtataggctgggaagtaaaatgctaaaggaatgagaaggcatttggggttgagttcaacctaagaggcaggggagccacagggaaagacctagcacctgccacagaagagaattaggaagcagaattgaactataagcaattttgaggtgttcgttgggctgcagttgaaatattttttgaggttaatgagacatttgaaatggccgtgtattgtttaactcttgcatagtcctgcatagggaacaatctaataggatttctctgtgaatcaagtcttagaaatttgcttttaatttttatgaaaaacgcccatttctttgtttttgagacagagtcctgctctgtcatccaggctgggttgcagtggcgtgatcttggcccactgcaatctctgcctcctgggttcaggcaattttcctgtctcagcctcccgagtagctgggatttcaagtgcctgccaccatgcccggctaaatttttttgtatttttggtacagatggagtatcaccatgttggccaggctggtctcgaactcctgacctcaagtgattcaccagccttgacctcccaaagtgttgggatcacaggcatgagccactgtgcctgtgccccaaaacaccaatttctgatgtgtgatgcatgtaagatagaacaaacttcagtaaagcggggacttgaaaagaggctttggtaacagctgtcagcattaacccttgcccctccgtacctcctaatcccacccctgctcaaagtatgttcatctgagaatttgtctccataactatgtgactataaaaattctcatcgattttgttagttgatcaattgagggaaaaacatatgttacttgatataactggtgggtcaaaagaattaacccaggcaaatttgagataggtggatgggatgatggattgaaaatacagctgctctctttccaatcatgtactaagtaatttgggaaagattgatctaattgggtctagagagtacacttcacatggcattgtttgactttttttctgcatcgctagcgatctgtgcattacaactcaaatcagtcgggtttcctggcatatgtaattgccaatgttttttaccagaagagaaacattactcccacctcttcttattatgttacaaactatagtgctaatgaccatcgaccaacagtgactttcaggatgacctgtgtgagttttatctgaaaccatgtgaatttttcatcttaaaagtcccttagaatctcagtctatgtacactcaggtttgttgcaggtttagagttccgtgttttttgtttctaatgtagacacagccttataatttacaacagcattcactaattaaaattgtaagcataattactatccacgatacttattattagtttgcattcataaagctcaaaattcacttcatcctttcaagtagtgaataattagtttctttgggtttgcagctttatcatccttttatgacccatttggaagaaataaacaaccaaccccctggaagactgctttaaaaagctggaaatacattgtccagctagtacaatgaggctaatacaatgtggaaaatattacttttctttgattttagtagcctgtttatctttacatttactgaacaaataactattgagcacctaatgtatactgggacccttggggaggcaaagatgaatcaaagattctgtccttaaagaccttaagttaagacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgattaattaacaggactgaccgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacaggatccactagtaacggccgccagtgtgctggaattccgaggtcgacggtatcgataagggcgcgcctgcggcaagggagttgcgggcgcacaggtaaggcgcgggaccgggaagggggcggcgccccgggatcggtctagggcggcggaggctgccggggcgggggtgtgtctgggtgtggccccggcgcgcgcggggtcctgagggaggggctcgccaagggggcgggcccgcgagctcgacagccccgagccaaaggggcggaaggtcgccgagtgctgggaggggaggtggggcagcgactgagcgccgcaggagctggctgcaacccagacgcggcccgggagcgtcgggtatgagctaacggggcgggggtgcggggaacgagcccagtcaaggagcagcacctacctccgggggtggaaatgggccggaatgggccggaacaccgtcccggaagtgaaaccccccgccccctcctccgcagcgagcgacgtgccggaaggaaatcagtcaccctccccccgcccctccccccagcactttccttgcaggagccccctcccctcagcctgtactcctcagggaggcggagggcgggcctatcgtgcccgcggcaactccgccctccgggtctcgggggcgtggccagagggaaagttttgtgggcctgaagaaggtggggcttgaggaggagtctgagggcgtgtgaggggcggggtttaagtggacgcagcaagcagctttgcgtcttgacgtcacgaagaccttatatactcgactcagccgccatctcagcctttcggagctgtgcggtgtttaaaccgccacc

SEQ ID NO: 55 - DNA, 10872 bp, Artificial
  pIM-RAG1-Hygro
ggcgcgccagatctgtacattcgaagatatcttaattaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaacggatccgcagcctctttcccacccaccttgggactcagttctgccccagatgaaattcagcacccacatattaaattttcagaatggaaatttaagctgttccgggtgagatcctttgaaaagacacctgaagaagctcaaaaggaaaagaaggattcctttgaggggaaaccctctctggagcaatctccagcagtcctggacaaggctgatggtcagaagccagtcccaactcagccattgttaaaagcccaccctaagttttcgaagaaatttcacgacaacgagaaagcaagaggcaaagcgatccatcaagccaaccttcgacatctctgccgcatctgtgggaattcttttagagctgatgagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttcccgaggaacgtgaccatggagtggcacccccacacaccatcctgtgacatctgcaacactgcccgtcggggactcaagaggaagagtcttcagccaaacttgcagctcagcaaaaaactcaaaactgtgcttgaccaagcaagacaagcccgtcagcacaagagaagagctcaggcaaggatcagcagcaaggatgtcatgaagaagatcgccaactgcagtaagatacatcttagtaccaagctccttgcagtggacttcccagagcactttgtgaaatccatctcctgccagatctgtgaacacattctggctgaccctgtggagaccaactgtaagcatgtcttttgccgggtctgcattctcagatgcctcaaagtcatgggcagctattgtccctcttgccgatatccatgcttccctactgacctggagagtccagtgaagtcctttctgagcgtcttgaattccctgatggtgaaatgtccagcaaaagagtgcaatgaggaggtcagtttggaaaaatataatcaccacatctcaagtcacaaggaatcaaaagagatttttgtgcacattaataaagggggtcgagtaacgcgtgcaggcatgcaagctggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgtaagtgatatctactagatttatcaaaaagagtgttgacttgtgagcgctcacaattgatacttagattcatcgagagggacacgtcgactactaaccttcttctctttcctacagctgagatcaccggcgaaggagggccaccatggcttcttaccctggacaccagcatgcttctgcctttgaccaggctgccagatccaggggccactccaacaggagaactgccctaagacccagaagacagcaggaagccactgaggtgaggcctgagcagaagatgccaaccctgctgagggtgtacattgatggacctcatggcatgggcaagaccaccaccactcaactgctggtggcactgggctccagggatgacattgtgtatgtgcctgagccaatgacctactggagagtgctaggagcctctgagaccattgccaacatctacaccacccagcacaggctggaccagggagaaatctctgctggagatgctgctgtggtgatgacctctgcccagatcacaatgggaatgccctatgctgtgactgatgctgttctggctcctcacattggaggagaggctggctcttctcatgcccctccacctgccctgaccctgatctttgacagacaccccattgcagccctgctgtgctacccagcagcaaggtacctcatgggctccatgaccccacaggctgtgctggcttttgtggccctgatccctccaaccctccctggcaccaacattgttctgggagcactgcctgaagacagacacattgacaggctggcaaagaggcagagacctggagagagactggacctggccatgctggctgcaatcagaagggtgtatggactgctggcaaacactgtgagatacctccagtgtggaggctcttggagagaggactggggacagctctctggaacagcagtgccccctcaaggagctgagccccagtccaatgctggtccaagaccccacattggggacaccctgttcaccctgttcagagcccctgagctgctggctcccaatggagacctgtacaatgtgtttgcctgggctctggatgttctagccaagaggctgaggtccatgcatgtgttcatcctggactatgaccagtcccctgctggatgcagagatgctctgctgcaactaacctctggcatggtgcagacccatgtgaccacccctggcagcatccccaccatctgtgacctagccagaacctttgccagggagatgggagaggccaactaaacctgagctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatccatttttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgacccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccaatattaaacttgatgagctctagagatggtcatgcattttaaaaagaattactcaaaatattgtcttggaataccagagagcaagtgctttaagtataggctgggaagtaaaatgctaaaggaatgagaaggcatttggggttgagttcaacctaagaggcaggggagccacagggaaagacctagcacctgccacagaagagaattaggaagcagaattgaactataagcaattttgaggtgttcgttgggctgcagttgaaatattttttgaggttaatgagacatttgaaatggccgtgtattgtttaactcttgcatagtcctgcatagggaacaatctaataggatttctctgtgaatcaagtcttagaaatttgcttttaatttttatgaaaaacgcccatttctttgtttttgagacagagtcctgctctgtcatccaggctgggttgcagtggcgtgatcttggcccactgcaatctctgcctcctgggttcaggcaattttcctgtctcagcctcccgagtagctgggatttcaagtgcctgccaccatgcccggctaaatttttttgtatttttggtacagatggagtatcaccatgttggccaggctggtctcgaactcctgacctcaagtgattcaccagccttgacctcccaaagtgttgggatcacaggcatgagccactgtgcctgtgccccaaaacaccaatttctgatgtgtgatgcatgtaagatagaacaaacttcagtaaagcggggacttgaaaagaggctttggtaacagctgtcagcattaacccttgcccctccgtacctcctaatcccacccctgctcaaagtatgttcatctgagaatttgtctccataactatgtgactataaaaattctcatcgattttgttagttgatcaattgagggaaaaacatatgttacttgatataactggtgggtcaaaagaattaacccaggcaaatttgagataggtggatgggatgatggattgaaaatacagctgctctctttccaatcatgtactaagtaatttgggaaagattgatctaattgggtctagagagtacacttcacatggcattgtttgactttttttctgcatcgctagcgatctgtgcattacaactcaaatcagtcgggtttcctggcatatgtaattgccaatgttttttaccagaagagaaacattactcccacctcttcttattatgttacaaactatagtgctaatgaccatcgaccaacagtgactttcaggatgacctgtgtgagttttatctgaaaccatgtgaatttttcatcttaaaagtcccttagaatctcagtctatgtacactcaggtttgttgcaggtttagagttccgtgttttttgtttctaatgtagacacagccttataatttacaacagcattcactaattaaaattgtaagcataattactatccacgatacttattattagtttgcattcataaagctcaaaattcacttcatcctttcaagtagtgaataattagtttctttgggtttgcagctttatcatccttttatgacccatttggaagaaataaacaaccaaccccctggaagactgctttaaaaagctggaaatacattgtccagctagtacaatgaggctaatacaatgtggaaaatattacttttctttgattttagtagcctgtttatctttacatttactgaacaaataactattgagcacctaatgtatactgggacccttggggaggcaaagatgaatcaaagattctgtccttaaagaccttaagacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccccggggatcaagggcgaattctgcagatgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctacgcgaattcgcccttgcgcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcctta

SEQ ID NO: 56 - protein, 354 aa, artificial sequence
  DMD21 single chain meganuclease
MANTKYNEEFLLYLAGFVDGDGSIIAQIKPRQSYKFKHQLELTFTVGQKTQRRWFLDKLVDEIGVGYVTDSGSMSAYRLSKIKPLHNFLTQLQPFLELKQKQANLVLKIIEQLPSAKESPDKFLEVCTWVDQVAALNDSKTRKTTSETVRAVLDNLSEKKKSSPAAGGSDKYNQALSKYNQALSKYNQALSGGGGSNKKFLLYLAGFVDSDGSIIAQIRPNQSAKFKHYLQLTFQVTQKTQRRWFLDKLVDRIGVGYVRDSGSVSDYKLSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 57 - protein, 354 aa, artificial sequence
  CAPNS1 single chain meganuclease
MANTKYNEEFLLYLAGFVDGDGSIVAQIKPNQRAKFKHQLSLTFQVTQKTQRRWLLDKLVDEIGVGYVQDSGSVSNYRLSEIKPLHNFLTQLQPFLELKQKQANLVLKIIEQLPSAKESPDKFLEVCTWADQIAALNDSKTRKTTSETVRAVLDSLSEKKKPSPAAGGSDKYNQALSKYNQALSKYNQALSGGGGSNKKFLLYLAGFVDSDGSIIAQIKPRQSYKFKHQLRLTFYVTQKTQRRWFLDKLVDRIGVGYVEDSGSVSRYVLSEIKPLHNFLTQLQPFLKLKQKQANLVLKIIEQLPSAKESPDKFLEVCTWVDQVAALNDSKTRKTTSETVRAVLDSLSEKKKSSP

SEQ ID NO: 58 - DNA, 10613 bp, Artificial; identical to SEQ ID NO: 45
  pIM.RAG1.CMV.Neo

SEQ ID NO: 59 - DNA, 12415 bp, Artificial; identical to SEQ ID NO: 46
  pIM.RAG1.CMV.Neo.Luc

SEQ ID NO: 60 - DNA, 6089 bp, Artificial
  hsRAG1 meganuclease expression plasmid
atggccaataccaaatataacgaagagttcctgctgtacctggccggctttgtggacggtgacggtagcatcatcgctcagattaatccaaaccagtcttctaagtttaaacatcgtctacgtttgaccttttatgtgactcaaaagacccagcgccgttggtttctggacaaactagtggatgaaattggcgttggttacgtacgtgattctggatccgtttcccagtacgttttaagcgaaatcaagccgctgcacaacttcctgactcaactgcagccgtttctggaactgaaacagaaacaggcaaacctggttctgaaaattatcgaacagctgccgtctgcaaaagaatccccggacaaattcctggaagtttgtacctgggtggatcagattgcagctctgaacgattctaagacgcgtaaaaccacttctgaaaccgttcgtgctgtgctggacagcctgagcgggaagaagaaatcctccccggcggccggtggatctgataagtataatcaggctctgtctaaatacaaccaagcactgtccaagtacaatcaggccctgtctggtggaggcggttccaacaaaaagttcctgctgtatcttgctggatttgtggattctgatggctccatcattgctcagataaaaccacgtcaatctaacaagttcaaacaccagctctccttgacttttgcagtcactcagaagacacaaagaaggtggttcttggacaaattggttgataggattggtgtgggctatgtctatgacagtggctctgtgtcagactaccgcctgtctgaaattaagcctcttcataactttctcacccaactgcaacccttcttgaagctcaaacagaagcaagcaaatctggttttgaaaatcatcgagcaactgccatctgccaaggagtcccctgacaagtttcttgaagtgtgtacttgggtggatcagattgctgccttgaatgactccaagaccagaaaaaccacctctgagactgtgagggcagttctggatagcctctctgagaagaaaaagtcctctccttagtctagagggcccgcggttcgaaggtaagcctatccctaaccctctcctcggtctcgattctacgcgtaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgacc

(19) United States
(12) Patent Application Publication
(10) Pub. No.: US 2013/0045539 A1
(43) Pub. Date: Feb. 21, 2013

(54) MEGANUCLEASE RECOMBINATION SYSTEM

(71) Applicants: Christophe Delenda, Paris (FR); Jean-Pierre Cabaniols, Saint Leu La Foret (FR)

(72) Inventors: Christophe Delenda, Paris (FR); Jean-Pierre Cabaniols, Saint Leu La Foret (FR)

(73) Assignee: CELLECTIS, Paris (FR)

(21) Appl. No.: 13/579,799

(22) PCT Filed: Feb. 18, 2011
(86) PCT No.: PCT/IB11/50682

§ 371 Date: Oct. 25, 2012

(51) Int. Cl. C12N 15/90; C12N 5/071; C12N 5/09; C12N 15/85

(52) U.S. Cl. 435/462; 435/320.1; 435/358; 435/369; 435/371; 435/372; 435/357; 435/354

(57) ABSTRACT

The invention relates to a set of genetic constructs which comprises at least a first recombinogenic construct (i) with at least two portions homologous to the genomic regions preceding and following the DNA target site of a site specific endonuclease and also comprising both a negative selection and positive selection mark interposed with the homologous portions as well as a region into which a sequence of interest can be cloned adjacent to the positive selection marker; and a second construct (ii, iii or iv) comprising the meganuclease. The present invention also relates to a kit comprising these constructs and methods to use this set of constructs to introduce into the genome of a target cell, tissue or organism a sequence of interest.

The present invention relates to a set of reagents to allow the introduction of a DNA sequence into a specific site in the genome of a target cell. In particular this DNA sequence encodes a gene and is introduced into the target cell via an induced homologous recombination (HR) event. The present invention also relates to a set of genetic constructs comprising at least two portions homologous to portions flanking a genomic target site for a meganuclease and a positive selection marker and a negative selection marker; as well as improved methods to introduce a DNA sequence into the genome of a target cell.

Since the first gene targeting experiments in yeast more than 25 years ago (Hinnen et al, 1978; Rothstein, 1983), homologous recombination (HR) has been used to insert, replace or delete genomic sequences in a variety of cells (Thomas and Capecchi, 1987; Capecchi, 2001; Smithies, 2001). Targeted events occur at a very low frequency in mammalian cells, making the use of innate HR impractical. The frequency of homologous recombination can be significantly increased by a specific DNA double-strand break (DSB) at a locus (Rouet et al, 1994; Choulika et al, 1995). Such DSBs can be induced by meganucleases, sequence-specific endonucleases that recognize large DNA recognition target sites (12 to 30 bp).

Meganucleases show high specificity to their DNA target, these proteins can cleave a unique chromosomal sequence and therefore do not affect global genome integrity. Natural meganucleases are essentially represented by homing endonucleases, a widespread class of proteins found in eukaryotes, bacteria and archae (Chevalier and Stoddard, 2001). Early studies of the I-SceI and HO homing endonucleases have illustrated how the cleavage activity of these proteins can be used to initiate HR events in living cells and have demonstrated the recombinogenic properties of chromosomal DSBs (Dujon et al, 1986; Haber, 1995). Since then, meganuclease-induced homologous recombination has been successfully used for genome engineering purposes in bacteria (Posfai et al, 1999), mammalian cells (Sargent et al, 1997; Donoho et al, 1998; Cohen-Tannoudji et al, 1998), mice (Gouble et al, 2006) and plants (Puchta et al, 1996; Siebert and Puchta, 2002).

More recently, TAL effector endonucleases (TALEN) have been engineered to recognize and cleave a DNA target with high specificity. These TALEN comprise a TAL (Transcription Activator-Like) effector DNA domain fused to a nuclease domain (e.g; FokI) (Christian et al, 2010).

A further class of nucleases can also be used to cleave a genomic target and so induce a DSB, this further class of nucleases are called Zinc-finger nucleases (ZFNs) and are artificial restriction enzymes generated by fusing a zinc finger DNA-binding domain to a DNA-cleavage domain. In a similar fashion to TALs, Zinc finger domains can be engineered so as to target any DNA sequence (Kim et al, 1996).

Even with the increasing availability of materials which can induce DSBs at a specific point in the genome of a target cell, efforts to develop methods and materials to routinely and reproducible transform a population of target cells have not yet been developed. A number of reasons exist for this including the inherent complexity of a prokaryotic or more particularly a eukaryotic genome. Workers have increasingly found that the genome has a remarkable capacity to resist damage, which is what a DSB essentially is. In addition the technical limitations which apply to all transformation methods namely the ability to routinely identify a rare transformant out of a background population of non-transformed cells continue to present problems in the generation of transformation methods.

A method to harness the potential of HR in introducing a sequence of interest into any point in the genome of a target cell or organism, so allowing more detailed genomic manipulations than ever before possible is provided.

The inventors have now developed a new set of genetic constructs comprising:

a) Construct (i) encoded by a nucleic acid molecule, which comprises at least the following components:

(N)ₙ-HOMO1-P-M-HOMO2-(N)ₘ (i)

wherein n and m are integer and represent 0 or 1, with the proviso that when n=1, m=0 and when n=0, m=1; thus component N can be disposed either before HOMO1 or after HOMO2, and components P and M can be disposed in the order P-M or M-P between HOMO1 and HOMO2;

wherein N comprises the components (PROM1)-(NEG)-(TERM1); P comprises the components (PROM2)-(POS)-(TERM2) and M comprises the components (PROM3)-(MCS)-(TERM3); and

wherein PROM1 is a first transcriptional promoting sequence; NEG is a negative selection marker; TERM1 is a first transcriptional termination sequence; HOMO1 is a portion homologous to a genomic portion preceding a nuclease DNA target sequence; PROM2 is a second transcriptional promoting sequence; POS is a positive selection marker; TERM2 is a second transcriptional termination sequence; PROM3 is a third transcriptional promoting sequence; MCS is a multiple cloning site, where a gene of interest (GOI) may be inserted; TERM3 is a third transcriptional termination sequence; HOMO2 is a portion homologous to a genomic portion following said DNA target sequence of a meganuclease, TALEN or ZFN;

b) At least one construct selected from the group comprising, constructs (ii) or (iii) encoded by nucleic acid molecules, which comprise at least one of the following components:

PROM4-NUC1 (ii);

NUC2 (iii); or

this set also comprises sequence (iv) which is an isolated or recombinant protein which comprises at least the following component:

NUC3 (iv);

wherein PROM4 is a fourth transcriptional promoting sequence; NUC1 is the open reading frame (ORF) of a meganuclease, a TALEN or a ZFN; NUC2 is a messenger RNA (mRNA) version of said meganuclease, TALEN or ZFN; NUC3 is an isolated or recombinant protein of said meganuclease, TALEN or ZFN;

wherein said meganuclease, said TALEN or said ZFN from constructs (ii) or (iii) or sequence (iv) recognize and cleave said DNA target sequence; and wherein constructs (ii) or (iii) or sequence (iv) are configured to be co-transfected with construct (i) into at least one target cell.

More generally, any nuclease able to specifically cleave a genomic target and so induce a DSB and having a double-stranded DNA target sequence of 12 to 45 bp can be used in the present invention. Non-limitating examples of nucleases encompassed by the present invention, are meganucleases, TALEN, ZFN, but the present invention could also work with chimeric endonucleases defined as any fusion protein comprising at least one endonuclease able to cleave a genomic target and so induce a DSB and having a double-stranded DNA target sequence of 12 to 45 bp.

In addition to nucleases which can induce a DSB at a specific genomic target, the present invention also encompasses the use of nucleases that can induce a single strand break (SSB) at a specific genomic target sequence of between 12 to 45 bp. A SSB is also known as a nick and such nicking nucleases are explicitly encompassed within the present invention.

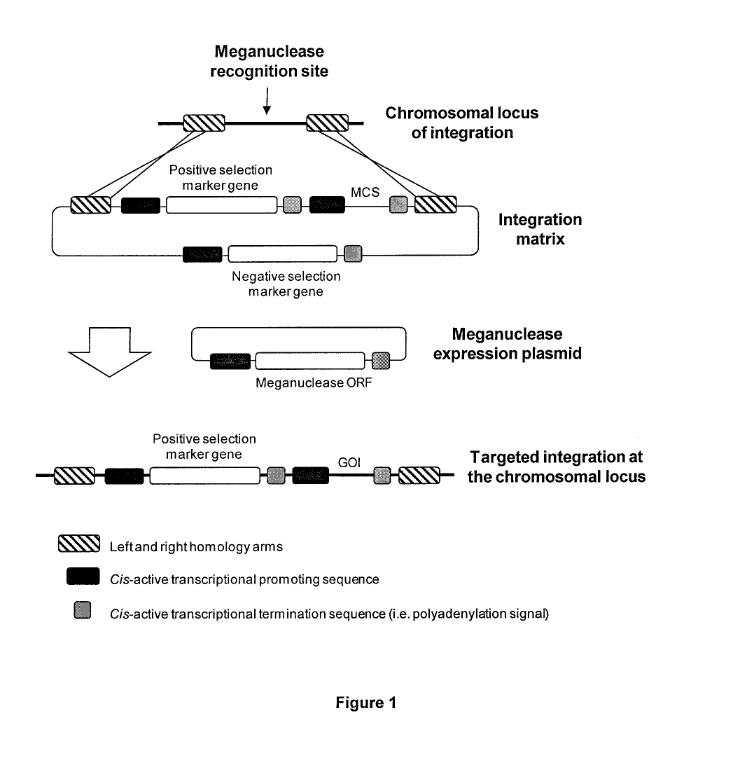

Constructs according to the present invention are illustrated in a non-limitative way in FIG. 1, the integration matrix [construct (i)] and the nuclease expression plasmid [construct (ii)] are co-transfected into cells. Upon co-transfection, the engineered nuclease is expressed, recognizes its endogenous recognition site, binds to it and induces a DNA double-strand break at this precise site.

The cell senses the DNA damage and triggers homologous recombination to fix it, using the co-transfected integration matrix as a DNA repair matrix since it contains regions homologous surrounding the broken DNA. The positive selection marker (POS) and the GOI, which are cloned in the integration matrix in between the homology regions, get integrated at the meganuclease recognition site during this recombination event. Thus, stable targeted cell clones can be selected for the drug resistance and expression of the recombinant protein of interest.

Examples of the types of genetic elements that can be used in constructs according to the present invention are provided below. These examples are illustrative only and should not be considered to restrict the scope of the invention in any way.

A list of positive and negative selection marker genes is provided in Table I below.

TABLE I

| | |
|---|---|
| Examples of | Neomycin phosphotransferase resistant gene, nptl (G418 |
| positive | geneticin) |
| marker | Hygromycin phosphotransferase resistant gene, hph |
| genes | (hygromycin B) |
| | Puromycin N-acetyl transferase gene, pac (puromycin) |
| | Blasticidin S deaminase resistant gene, bsr (blasticidin) |
| | Bleomycin resistant gene, sh ble (zeocin, phleomycin, |
| | bleomycin) |
| Examples of | Thymidine kinase from herpes simplex virus, HSV TK |
| marker | (ganciclovir) |
| genes | Cytosine deaminase coupled to uracyl phosphoribosyl |
| | transferase, CD:UPRT (5-fluorocytosine) |

Table II below provides a list of cis-active promoting sequences. Depending on the intrinsic transcriptional specificity of each dedicated cell type, various promoting sequences and/or internal ribosome entry sites (IRES) can be used for driving the expression of (i) custom meganuclease open reading frames, (ii) selection marker genes and genes of interest (GOIs). In addition to the examples given in this table, additional cis-active regulatory sequences can also be inserted in meganuclease expression plasmids and integration matrices in order to emphasize the transcriptional expression level (i.e. enhancers) and/or to reduce susceptible transcriptional silencing [i.e. silencers such as scaffold/matrix attachment regions (S/MARs)].

TABLE II

| | |
|---|---|
| Examples of | Cytomegalovirus immediate-early promoter |
| constitutive | (pCMV) |
| promoting sequences | Simian virus 40 promoter (pSV40) |
| | Human elongation factor 1α promoter (phEF1α) |
| | Human phosphoglycerate kinase promoter |
| | (phPGK) |
| | Murine phosphoglycerate kinase promoter |
| | (pmPGK) |
| | Human polyubiquitin promoter (phUbc) |
| | Thymidine kinase promoter from human herpes |
| | simplex virus (pHSV-TK) |
| | Human growth arrest specific 5 promoter |
| | (phGAS5) |
| Example of inducible | Tetracycline-responsive element (pTRE) |
| promoting sequences | |
| Examples of internal | IRES sequence from encephalopathy myocarditis |
| ribosome entry sites | virus (IRES EMCV) |
| (IRES) | IRES sequence from foot and mouth disease virus |
| | (IRES FMDV) |

Table III provides a list of various tag elements, these different types of tag sequences can be inserted in multiple cloning sites (MCS) of integration matrices in order to dispose of N-terminal and C-terminal fusions after GOI cloning.

TABLE III

| | |
|---|---|
| Examples of tags | FLAP |
| used for imaging | SNAP, CLIP |
| | ACP, MCP |
| | IQ |
| Examples of tags | Histidine |
| used for purification | STREP |
| | SBP, CBP |
| Examples of tags | HA |
| used for | c-myc |
| immunodetection | V5 |
| Examples of tags used | NLS |
| for cellular addressing | |

Table IV provides a list of the most commonly used reporter genes. Different types of reporter genes can be introduced in integration matrices (in place of the GOI, at the MCS sequence) in order to dispose of positive controls.

TABLE IV

| | |
|---|---|
| Examples | [symbol] Living color [symbol] genes, i.e. encoding green fluorescent protein |
| of reporter | (GFP), red fluorescent protein (RFP) . . . |
| genes | Luciferase genes (firefly, renilla) |
| | β-galactosidase gene (LacZ) |
| | Human secreted alkaline phosphatase gene (hSEAP) |
| | Murine secreted alkaline phosphatase gene (mSEAP) |

In the present invention, a transcriptional promoting sequence is a nucleotide sequence which when placed in combination with a second nucleotide sequence encoding an open reading frame causes the transcription of the open reading frame. In addition in the case of a RNA molecule, a promoter can also refer to a non-coding sequence which acts to increase the levels of translation of the RNA molecule.

In the present invention, a transcriptional termination sequence is a nucleotide sequence which when placed after a nucleotide sequence encoding an open reading frame causes the end of transcription of the open reading frame.

In the present invention, a homologous portion refers to a nucleotide sequence which shares nucleotide residues in common with another nucleotide sequence so as to lead to a homologous recombination between these sequences, more particularly having at least 95% identity, preferably 97% identity and more preferably 99% identity. The first and second homologous portions of construct (i) (HOMO1 and HOMO2) can be 100% identical or less as indicated to the sequences flanking the nuclease, such as meganuclease, TALEN or the ZFN, target DNA sequence in the target cell genome.

In particular the overlap between the portions HOMO1 and HOMO2 from construct (i) and the homologous portions from the host cell genome is at least 200 bp and no more than 6000 bp, preferably this overlap is between 1000 bp and 2000 bp.

In particular therefore components HOMO1 and HOMO2 from construct (i), comprise at least 200 bp and no more than 6000 bp of sequence homologous to the host cell genome respectively.

Most particularly components HOMO1 and HOMO2 from construct (i), comprise at least 1000 bp and no more than 2000 bp of sequence homologous to the host cell genome respectively.

The amounts of overlap necessary to allow efficient levels of homologous recombination are known in the art (Perez et al., (2005)); starting from these known levels the inventors have identified the most efficient ranges of overlap for use with the set of constructs according to the present invention.

In the present invention, a meganuclease target DNA site or meganuclease recognition site is intended to mean a 22 to 24 bp double-stranded palindromic, partially palindromic (pseudo-palindromic) or non-palindromic polynucleotide sequence that is recognized and cleaved by a LAGLIDADG homing endonuclease. These terms refer to a distinct DNA location, preferably a genomic location, at which a double stranded break (cleavage) is to be induced by the meganuclease.

The meganuclease target DNA site can be the DNA sequence recognized and cleaved by a wild type meganuclease such as I-CreI or I-DmoI.

Alternatively the meganuclease DNA target site can be the DNA sequence recognized and cleaved by altered meganucleases which recognize and cleave different DNA target sequences.

The making of functional chimeric meganucleases, by fusing the N-terminal I-DmoI domain with an I-CreI monomer (Chevalier et al., Mol. Cell., 2002, 10, 895-905; Epinat et al., Nucleic Acids Res, 2003, 31, 2952-62; International PCT Applications WO 03/078619 and WO 2004/031346) have also been described.

The inventors and others have shown that meganucleases can be engineered so as to recognize different DNA targets. The I-CreI enzyme in particular has been studied extensively and different groups have used a semi-rational approach to locally alter the specificity of I-CreI (Seligman et al., Genetics, 1997, 147, 1653-1664; Sussman et al., J. Mol. Biol., 2004, 342, 31-41; International PCT Applications WO 2006/097784, WO 2006/097853, WO 2007/060495 and WO 2007/049156; Arnould et al., J. Mol. Biol., 2006, 355, 443-458; Rosen et al., Nucleic Acids Res., 2006, 34, 4791-4800; Smith et al., Nucleic Acids Res., 2006, 34, e149), I-SceI (Doyon et al., J. Am. Chem. Soc., 2006, 128, 2477-2484), PI-SecI (Gimble et al., J. Mol. Biol., 2003, 334, 993-1008) and I-MsoI (Ashworth et al., Nature, 2006, 441, 656-659).

In addition, hundreds of I-CreI derivatives with locally altered specificity were engineered by combining the semi-rational approach and High Throughput Screening:
Residues Q44, R68 and R70 or Q44, R68, D75 and I77 of I-CreI were mutagenized and a collection of variants with altered specificity at positions±3 to 5 of the DNA target (5NNN DNA target) were identified by screening (International PCT Applications WO 2006/097784 and WO 2006/097853; Arnould et al., J. Mol. Biol., 2006, 355, 443-458; Smith et al., Nucleic Acids Res., 2006, 34, e149).
Residues K28, N30 and Q38 or N30, Y33, and Q38 or K28, Y33, Q38 and S40 of I-CreI were mutagenized and a collection of variants with altered specificity at positions±8 to 10 of the DNA target (10NNN DNA target) were identified by screening (Smith et al., Nucleic Acids Res., 2006, 34, e149; International PCT Applications WO 2007/060495 and WO 2007/049156).

Two different variants were combined and assembled in a functional heterodimeric endonuclease able to cleave a chimeric target resulting from the fusion of two different halves of each variant DNA target sequence (Arnould et al., precited; International PCT Applications WO 2006/097854 and WO 2007/034262).

Furthermore, residues 28 to 40 and 44 to 77 of I-CreI were shown to form two separable functional subdomains, able to bind distinct parts of a homing endonuclease half-site (Smith et al. Nucleic Acids Res., 2006, 34, e149; International PCT Applications WO 2007/049095 and WO 2007/057781).

The combination of mutations from the two subdomains of I-CreI within the same monomer allowed the design of novel chimeric molecules (homodimers) able to cleave a palindromic combined DNA target sequence comprising the nucleotides at positions±3 to 5 and ±8 to 10 which are bound by each subdomain (Smith et al., Nucleic Acids Res., 2006, 34, e149; International PCT Applications WO 2007/049095 and WO 2007/057781).

The method for producing meganuclease variants and the assays based on cleavage-induced recombination in mammal or yeast cells, which are used for screening variants with altered specificity are described in the International PCT Application WO 2004/067736; Epinat et al., Nucleic Acids Res., 2003, 31, 2952-2962; Chames et al., Nucleic Acids Res., 2005, 33, e178, and Arnould et al., J. Mol. Biol., 2006, 355, 443-458. These assays result in a functional LacZ reporter gene which can be monitored by standard methods.

The combination of the two former steps allows a larger combinatorial approach, involving four different subdomains. The different subdomains can be modified separately and combined to obtain an entirely redesigned meganuclease variant (heterodimer or single-chain molecule) with chosen specificity. In a first step, couples of novel meganucleases are combined in new molecules (“half-meganucleases”) cleaving palindromic targets derived from the target one wants to cleave. Then, the combination of such “half-meganucleases” can result in a heterodimeric species cleaving the target of interest. The assembly of four sets of mutations into heterodimeric endonucleases cleaving a model target sequence or a sequence from the human RAG1, XPC and HPRT genes have been described in Smith et al. (Nucleic Acids Res., 2006, 34, e149), Arnould et al., (J. Mol. Biol., 2007, 371, 49-65), and WO2008/059382 respectively. Other examples of meganucleases can be used in the present invention such as those cleaving a target in the human Duchenne Muscular Dystrophy (DMD21, SEQ ID NO 56) gene or a target in the human Calpain, small subunit1 (CAPNS1, SEQ ID NO 57) gene.

All such variant meganucleases and the variant DNA targets which they recognize and cleave, are included in the present patent application and any combination of a particular meganuclease and its target can be used as the meganuclease target sequence present in the target cell genome and flanked by the genomic portions homologous to HOMO1 and HOMO2 represented from construct (i).

Similarly, other nucleases such as TALENs and ZFNs can be engineered so as to recognize and cleave a specific DNA target sequence and are included in the present patent application and any combination of a particular nuclease such as TALENs and/or ZFNs and its target can be used as the nuclease target sequence present in the target cell genome and flanked by the genomic portions homologous to HOMO1 and HOMO2 represented from construct (i).

In the present invention a marker gene is a gene product which when expressed allows the differentiation of a cell or population of cells expressing the marker gene versus a cell or population of cells not expressing the marker gene.

A positive selection marker confers a property which restores or rescues a cell comprising it from a selection step such as supplementation with a toxin.

A negative selection marker is either inherently toxic or causes a cell comprising it to die following a selection step such as supplementation with a pro-toxin, wherein the negative marker acts upon the pro-toxin to form a toxin.

In addition to selection using cell viability, other means of selection are encompassed by the present invention such as cell sorting based upon marker gene expression.

In the present invention a multiple cloning site is a short segment of DNA which contains several restriction sites so as to allow the sub-cloning of a fragment of interest into the plasmid comprising the multiple cloning site.

In the present invention a meganuclease is intended to mean an endonuclease having a double-stranded DNA target sequence of 12 to 45 bp. This may be a wild type version of a meganuclease such as I-CreI or I-DmoI or an engineered version of one of these enzymes as described above or fusion proteins comprising portions of one or more meganuclease(s).

The inventors have shown that this system can work with a number of diverse model mammalian cell lines for a number of GOIs.

According to further aspects of the present invention component (POS) is selected from the group: neomycin phosphotransferase resistant gene, nptl (SEQ ID NO 3); hygromycin phosphotransferase resistant gene, hph (SEQ ID NO 4); puromycin N-acetyl transferase gene, pac (SEQ ID NO 5); blasticidin S deaminase resistant gene, bsr (SEQ ID NO 6); bleomycin resistant gene, sh ble (SEQ ID NO 7).

Preferably component (NEG) is selected from the group: Thymidine kinase gene of the herpes simplex virus deleted of CpG islands, HSV TK DelCpG (SEQ ID NO 8); cytosine deaminase coupled to uracyl phosphoribosyl transferase gene deleted of CpG islands, CD:UPRT DelCpG (SEQ ID NO 9).

Random in cellulo linearization of the integration matrix can lead to random integration of the construct into the host genome. If the linearization occurs within the negative marker and so inactivates its function, these random integration events would not be eliminated by the pro-drug treatment of cells.

According to a further aspect of the present invention therefore there is provided a version of construct (i) which comprises at least two (N) components. The presence of two negative selection expression cassettes on the integration matrix; one upstream of the HOMO1 region and one downstream of the HOMO2 region, overcomes this problem.

Preferably elements PROM1, PROM2, PROM3 and PROMO are selected from the group: cytomegalovirus immediate-early promoter, pCMV (SEQ ID NO 10); simian virus 40 promoter, pSV40 (SEQ ID NO 11); human elongation factor 1α promoter, phEF1α (SEQ ID NO12); human phosphoglycerate kinase promoter, phPGK (SEQ ID NO 13); murine phosphoglycerate kinase promoter, pmPGK (SEQ ID NO 14); human polyubiquitin promoter, phUbc (SEQ ID NO 15); thymidine kinase promoter from human herpes simplex virus, pHSV-TK (SEQ ID NO 16); human growth arrest specific 5 promoter, phGAS5 (SEQ ID NO 17); tetracycline-responsive element, pTRE (SEQ ID NO18); internal ribosomal entry site (IRES) sequence from encephalopathy myocarditis virus, IRES EMCV (SEQ ID NO 19), IRES sequence from foot and mouth disease virus, IRES FMDV (SEQ ID NO 20), SV40.

Preferably elements TERM1, TERM2, TERM3 and TERM4 is selected from the group: polyadenylation signal, SV40 pA (SEQ ID NO 21), bovine growth hormone polyadenylation signal, BGH pA (SEQ ID NO 22).

Preferably element MCS comprises an in frame peptide tag at its 5′ or 3′ end, wherein said peptide tag is selected from the group: FLAG (SEQ ID NO 23), FLASH/REASH (SEQ ID NO 24), IQ (SEQ ID NO 25), histidine (SEQ ID NO 26), STREP (SEQ ID NO 27), streptavidin binding protein, SBP (SEQ ID NO 28), calmodulin binding protein, CBP (SEQ ID NO 29), haemagglutinin, HA (SEQ ID NO 30), c-myc (SEQ ID NO 31), V5 tag sequence (SEQ ID NO 32), nuclear localization signal (NLS) from nucleoplasmin (SEQ ID NO 33), NLS from SV40 (SEQ ID NO 34), NLS consensus (SEQ ID NO 35), thrombin cleavage site (SEQ ID NO 36), P2A cleavage site (SEQ ID NO 37), T2A cleavage site (SEQ ID NO 38), E2A cleavage site (SEQ ID NO 39).

In addition to detectable peptide tags, nuclear localization signals and purification tags the MCS can also comprise other useful additional sequences such as cell penetrating peptides, peptides which chelate detectable compounds such as fluorophores or radionuclides.

According to a further specific aspect of the present invention the MSC may comprises a reporter gene selected from the group: firefly luciferase gene (SEQ ID NO 40), renilla luciferase gene (SEQ ID NO 41), β-galactosidase gene, LacZ (SEQ ID NO 42), human secreted alkaline phosphatase gene, hSEAP (SEQ ID NO 43), murine secreted alkaline phosphatase gene, mSEAP (SEQ ID NO 44). Such a version of construct (i) can be used as a positive control to determine the level of gene expression resulting from the insertion of such a reporter gene by HR using the set of constructs according to the present invention.

In particular construct (i) comprises SEQ ID NO: 45 or SEQ ID NO: 46.

According to a second aspect of the present invention there is provided a kit to introduce a sequence encoding a GOI into at least one cell, comprising the set of genetic constructs according to the first aspect of the present invention; and instructions for the generation of a transformed cell using said set of genetic constructs.

In particular said kit further comprises at least one target cell is selected from the group comprising: CHO-K1 cells; HEK293 cells; Caco2 cells; U2-OS cells; NIH 3T3 cells; NSO cells; SP2 cells; CHO-S cells; DG44 cells; K-562 cells, U-937 cells; MRC5 cells; IMR90 cells; Jurkat cells; HepG2 cells; HeLa cells; HT-1080 cells; HCT-116 cells; Hu-h7 cells; Huvec cells; Molt 4 cells.

According to a third aspect of the present invention there is provided a method for transforming by homologous recombination at least one cell comprising the steps of:

a) cloning a sequence coding for a gene of interest into position MCS of construct (i);

b) co-transfecting a target cell with said construct (i) of step a) and at least one of constructs (ii), (iii) or (iv) as defined here above;

c) selecting at least one cell based upon: the presence of component (POS) and the absence of component (NEG) from said target cell.

In particular wherein selection in step c) is carried out sequentially for the activity of the gene product encoded by (POS) and (NEG).

Alternatively the selection in step c) is carried out simultaneously for the activity of the gene product encoded by (POS) and (NEG).

### DEFINITIONS

Amino acid residues in a polypeptide sequence are designated herein according to the one-letter code, in which, for example, Q means Gln or Glutamine residue, R means Arg or Arginine residue and D means Asp or Aspartic acid residue.
Nucleotides are designated as follows: one-letter code is used for designating the base of a nucleoside: a is adenine, t is thymine, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a or t, v represents g, a or c, b represents g, t or c, h represents a, t or c, and n represents g, a, t or c.
by “meganuclease” is intended an endonuclease having a double-stranded DNA target sequence of 12 to 45 bp. Examples include I-Sce I, I-Chu I, I-Cre I-Csm I, PI-Sce I, PI-Tli I, PI-Mtu I, I-Ceu I, I-Sce II, I-Sce III, HO, PI-Civ I, PI-Ctr I, PI-Aae I, PI-Bsu I, PI-Dha I, PI-Dra I, PI-Mav I, PI-Mch I, PI-Mfu I, PI-Mfl I, PI-Mga I, PI-Mgo I, PI-Min I, PI-Mka I, PI-Mle I, PI-Mma I, PI-Msh I, PI-Msm I, PI-Mth I, PI-Mtu I, PI-Mxe I, PI-Npu I, PI-Pfu I, PI-Rma I, PI-Spb I, PI-Ssp I, PI-Fac PI-Mja I, PI-Pho I, PI-Tag I, PI-Thy I, PI-Tko I, PI-Tsp I, I-MsoI.
by “homodimeric LAGLIDADG homing endonuclease” is intended a wild-type homodimeric LAGLIDADG homing endonuclease having a single LAGLIDADG motif and cleaving palindromic DNA target sequences, such as I-CreI or I-MsoI or a functional variant thereof.
by “LAGLIDADG homing endonuclease variant” or “ZFN variant” or “TALEN variant” or “variant” is intended a protein obtained by replacing at least one amino acid of a LAGLIDADG homing endonuclease sequence or a TALEN sequence or a ZFN sequence respectively, with a different amino acid.
by “functional variant” is intended a LAGLIDADG homing endonuclease variant or a TALEN variant or a ZFN variant which is able to cleave a DNA target, preferably a new DNA target which is not cleaved by a wild type LAGLIDADG homing endonuclease or a TALEN or a ZFN variant. For example, such variants have amino acid variation at positions contacting the DNA target sequence or interacting directly or indirectly with said DNA target.
by “nuclease variant with novel specificity” is intended a variant having a pattern of cleaved targets (cleavage profile) different from that of the parent nuclease. The variants may cleave less targets (restricted profile) or more targets than the parent nuclease. Preferably, the variant is able to cleave at least one target that is not cleaved by the parent nuclease.

The terms “novel specificity”, “modified specificity”, “novel cleavage specificity”, “novel substrate specificity” which are equivalent and used indifferently, refer to the specificity of the variant towards the nucleotides of the DNA target sequence.
by “I-CreI” is intended the wild-type I-CreI having the sequence SWISSPROT P05725 or pdb accession code 1g9y.
by “domain” or “core domain” is intended the “LAGLIDADG homing endonuclease core domain” which is the characteristic αββαββα a fold of the homing endonucleases of the LAGLIDADG family, corresponding to a sequence of about one hundred amino acid residues. Said domain comprises four beta-strands folded in an antiparallel beta-sheet which interacts with one half of the DNA target. This domain is able to associate with another LAGLIDADG homing endonuclease core domain which interacts with the other half of the DNA target to form a functional endonuclease able to cleave said DNA target. For example, in the case of the dimeric homing endonuclease I-CreI (163 amino acids), the LAGLIDADG homing endonuclease core domain corresponds to the residues 6 to 94. In the case of monomeric homing endonucleases, two such domains are found in the sequence of the endonuclease; for example in I-DmoI (194 amino acids), the first domain (residues 7 to 99) and the second domain (residues 104 to 194) are separated by a short linker (residues 100 to 103).
by “subdomain” is intended the region of a LAGLIDADG homing endonuclease core domain which interacts with a distinct part of a homing endonuclease DNA target half-site. Two different subdomains behave independently or partly independently, and the mutation in one subdomain does not alter the binding and cleavage properties of the other subdomain, or does not alter it in a number of cases. Therefore, two subdomains bind distinct part of a homing endonuclease DNA target half-site.
by “beta-hairpin” is intended two consecutive beta-strands of the antiparallel beta-sheet of a LAGLIDADG homing endonuclease core domain which are connected by a loop or a turn,

by “single-chain meganuclease”, “single-chain chimeric meganucleave”, “single-chain meganuclease derivative”, “single-chain chimeric meganuclease derivative” or “single-chain derivative” is intended a meganuclease comprising two LAGLIDADG homing endonuclease domains or core domains linked by a peptidic spacer. The single-chain meganuclease is able to cleave a chimeric DNA target sequence comprising one different half of each parent meganuclease target sequence.

by “cleavage activity” the cleavage activity of the variant of the invention may be measured by a direct repeat recombination assay, in yeast or mammalian cells, using a reporter vector, as described in the PCT Application WO 2004/067736; Epinat et al., Nucleic Acids Res., 2003, 31, 2952-2962; Chames et al., Nucleic Acids Res., 2005, 33, e178, and Arnould et al., J. Mol. Biol., 2006, 355, 443-458. The reporter vector comprises two truncated, non-functional copies of a reporter gene (direct repeats) and a chimeric DNA target sequence within the intervening sequence, cloned in yeast or a mammalian expression vector. The DNA target sequence is derived from the parent homing endonuclease cleavage site by replacement of at least one nucleotide by a different nucleotide. Preferably a panel of palindromic or non-palindromic DNA targets representing the different combinations of the 4 bases (g, a, c, t) at one or more positions of the DNA cleavage site is tested (4ⁿ palindromic targets for n mutated positions). Expression of the variant results in a functional endonuclease which is able to cleave the DNA target sequence. This cleavage induces homologous recombination between the direct repeats, resulting in a functional reporter gene, whose expression can be monitored by appropriate assay.
by “DNA target”, “DNA target sequence”, “target sequence”, “target-site”, “target”, “site”; “recognition site”, “recognition sequence”, “homing recognition site”, “homing site”, “cleavage site” is intended a 22 to 24 bp double-stranded palindromic, partially palindromic (pseudo-palindromic) or non-palindromic polynucleotide sequence that is recognized and cleaved by a LAGLIDADG homing endonuclease. These terms refer to a distinct DNA location, preferably a genomic location, at which a double stranded break (cleavage) is to be induced by the endonuclease. The DNA target is defined by the 5′ to 3′ sequence of one strand of the double-stranded polynucleotide. Alternatively “DNA target”, “DNA target sequence”, “target sequence”, “target-site”, “target”, “site”; “recognition site”, “recognition sequence”, “homing recognition site”, “homing site”, “cleavage site” is intended a double-stranded palindromic, partially palindromic (pseudo-palindromic) or non-palindromic polynucleotide sequence that is recognized and cleaved by a nuclease such as a TALEN or ZFN.
by “DNA target half-site”, “half cleavage site” or half-site” is intended the portion of the DNA target which is bound by each nuclease domain such as LAGLIDADG homing endonuclease core domain or each TAL or each Zinc Finger domain.
by “chimeric DNA target” or “hybrid DNA target” is intended the fusion of a different half of two parent nuclease target sequences. In addition at least one half of said target may comprise the combination of nucleotides which are bound by separate subdomains (combined DNA target) in the case of a LAGLIDADG homing endonuclease target.
by “mutation” is intended the substitution, the deletion, and/or the addition of one or more nucleotides/amino acids in a nucleic acid/amino acid sequence.

by “nuclease” it is intended to mean any naturally occurring or artificial enzyme, molecule or other means which can cleave a specific genomic DNA target and so induce a DSB or SSB and having a double-stranded DNA target sequence of between 12 to 45 bp.
by “homologous” is intended a sequence with enough identity to another one to lead to a homologous recombination between sequences, more particularly having at least 95% identity, preferably 97% identity and more preferably 99%.
“Identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default settings.
“individual” includes mammals, as well as other vertebrates (e.g., birds, fish and reptiles). The terms “mammal” and “mammalian”, as used herein, refer to any vertebrate animal, including monotremes, marsupials and placental, that suckle their young and either give birth to living young (eutharian or placental mammals) or are egg-laying (metatharian or nonplacental mammals). Examples of mammalian species include humans and other primates (e.g., monkeys, chimpanzees), rodents (e.g., rats, mice, guinea pigs) and ruminants (e.g., cows, pigs, horses).
“gene of interest” or “GUI” refers to any nucleotide sequence encoding a known or putative gene product.
“genetic disease” refers to any disease, partially or completely, directly or indirectly, due to an abnormality in one or several genes. Said abnormality can be a mutation, an insertion or a deletion. Said mutation can be a punctual mutation. Said abnormality can affect the coding sequence of the gene or its regulatory sequence. Said abnormality can affect the structure of the genomic sequence or the structure or stability of the encoded mRNA. This genetic disease can be recessive or dominant. Such genetic disease could be, but are not limited to, cystic fibrosis, Huntington's chorea, familial hypercholesterolemia (LDL receptor defect), hepatoblastoma, Wilson's disease, congenital hepatic porphyrias, inherited disorders of hepatic metabolism, Lesch Nyhan syndrome, sickle cell anemia, thalassaemias, xeroderma pigmentosum, Fanconi's anemia, retinitis pigmentosa, ataxia telangiectasia, Bloom's syndrome, retinoblastoma, Duchenne's muscular dystrophy, and Tay-Sachs disease.
“vectors”: a vector which can be used in the present invention for instance as construct (ii) or (iii) as defined above includes, but is not limited to, a viral vector, a plasmid, a RNA vector or a linear or circular DNA or RNA molecule which may consists of a chromosomal, non chromosomal, semi-synthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available.

Viral vectors include retrovirus, adenovirus, parvovirus (e.g. adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosissarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996). The term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors. A vector according to the present invention comprises, but is not limited to, a YAC (yeast artificial chromosome), a BAC (bacterial artificial), a baculovirus vector, a phage, a phagemid, a cosmid, a viral vector, a plasmid, a RNA vector or a linear or circular DNA or RNA molecule which may consist of chromosomal, non chromosomal, semi-synthetic or synthetic DNA. In general, expression vectors of utility in recombinant DNA techniques are often in the form of “plasmids” which refer generally to circular double stranded DNA loops which, in their vector form are not bound to the chromosome. Large numbers of suitable vectors are known to those of skill in the art.

Vectors can comprise selectable markers, for example: neomycin phosphotransferase, histidinol dehydrogenase, dihydrofolate reductase, hygromycin phosphotransferase, herpes simplex virus thymidine kinase, adenosine deaminase, glutamine synthetase, and hypoxanthine-guanine phosphoribosyl transferase for eukaryotic cell culture; TRP1 for S. cerevisiae; tetracycline, rifampicin or ampicillin resistance in E. coli. These selectable markers can also be used as a part of the constructs (i) and (ii) according to the present invention.

Preferably said vectors are expression vectors, wherein a sequence encoding a polypeptide of the invention is placed under control of appropriate transcriptional and translational control elements to permit production or synthesis of said protein. Therefore, said polynucleotide is comprised in an expression cassette. More particularly, the vector comprises a replication origin, a promoter operatively linked to said encoding polynucleotide, a ribosome site, an RNA-splicing site (when genomic DNA is used), a polyadenylation site and a transcription termination site. It also can comprise enhancer or silencer elements. Selection of the promoter will depend upon the cell in which the polypeptide is expressed.

For a better understanding of the invention and to show how the same may be carried into effect, there will now be shown by way of example only, specific embodiments, methods and processes according to the present invention with reference to the accompanying drawings in which:

FIG. 1: Schematic representation of the meganuclease-mediated targeted integration process. The integration matrix and the meganuclease expression plasmid are co-transfected into eukaryotic cells. Upon co-transfection, the engineered meganuclease is expressed, recognizes its endogenous recognition site, binds to it and induces a DNA double-strand break at this precise site. The cell senses the DNA damage and triggers homologous recombination to fix it, using the co-transfected integration matrix (used as a DNA repair matrix since it contains regions homologous surrounding the broken DNA). The selection marker and the gene of interest (GOI) which has been cloned in the multiple cloning site (MCS) of the integration matrix in between the homology regions, get integrated at the meganuclease recognition site during this recombination event.

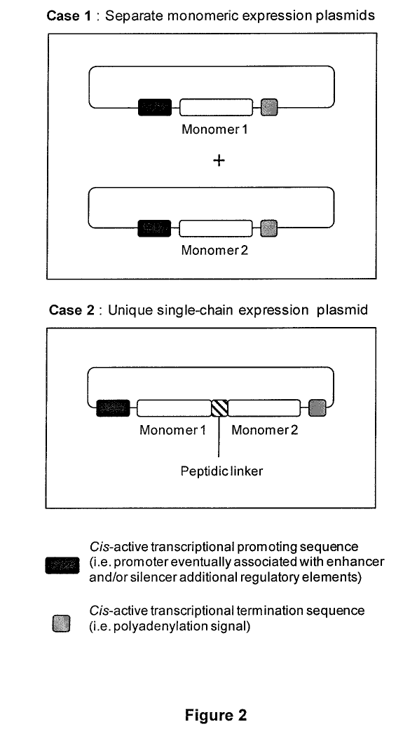

FIG. 2: Description of meganuclease-encoding plasmid(s). Two different strategies can be exploited for driving the expression of meganuclease monomeric sub-units, i.e. by introducing the open reading frame of each monomer in two separate plasmids (case 1) or in a unique plasmid wherein monomeric sub-units are expressed in a single-chain version (case 2).

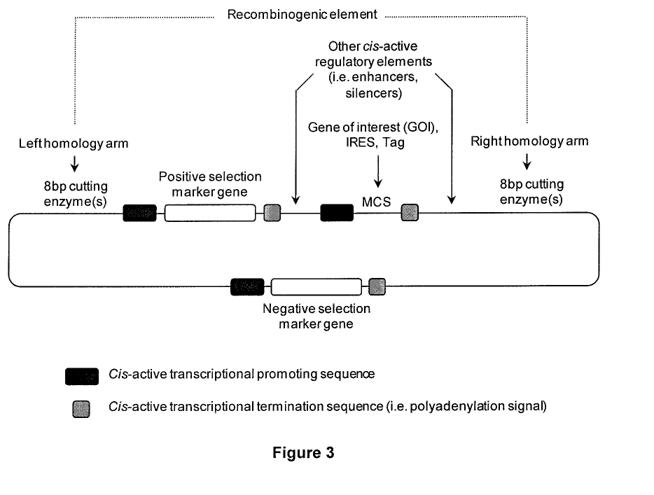

FIG. 3: Description of universal integration matrices. Schematic representation of the different genetic elements introduced in universal integration matrices. First, positive and selection marker genes are added in two different places: the former inserted in and the latter inserted out of the recombinogenic element. Second, different restriction sites have been introduced: 8 bp cutting sites for the cloning of left and right homology arms for any type of integration locus, a multiple cloning site (MCS) for the insertion of any GOI and other restriction sites in the case of additional element cloning (i.e. enhancers, silencers).

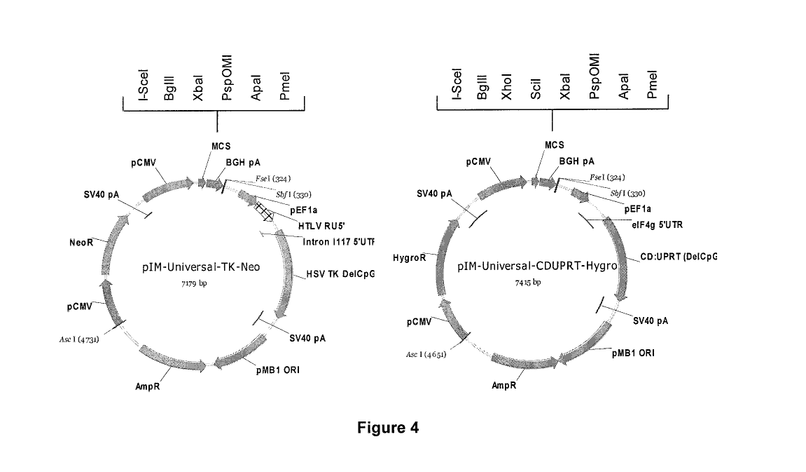

FIG. 4: Universal integration plasmid maps. Two examples of universal integration matrices are given by changing the type of positive [i.e. neomycin (NeoR) and hygromycin (HygroR) as examples] and negative (i.e. HSV TK DelCpG and CD:UPRT DelCpG) selection marker genes. Multiple cloning sites (MCS) are indicated for the cloning of the gene of interest (GOI). These plasmid backbones are universal in the sense that they can serve for HR in any type of chromosomal locus, by inserting the left homology arm at the AscI site and the right homology arm at FseI or SbfI site. The choice for such 8 bp cutters has been privileged over classical 6 bp cutters to reduce the possibility to find sites in the desired chromosomal regions to be amplified.

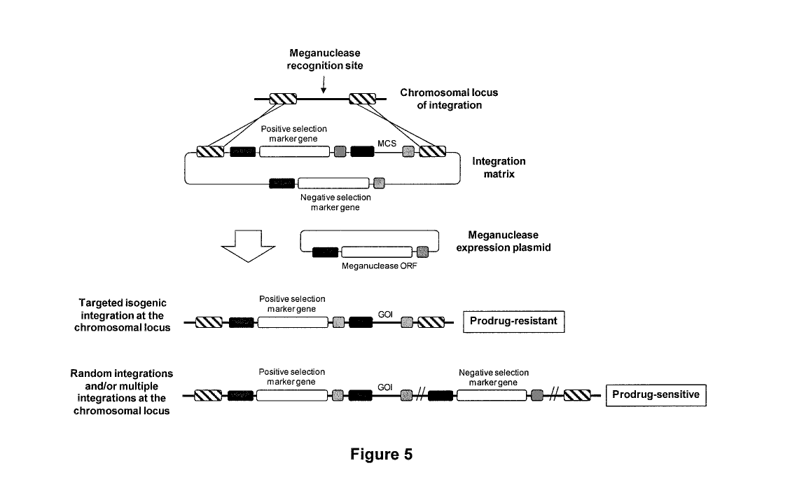

FIG. 5: Schematic representation of the meganuclease-mediated targeted integration process (counter selection). After a positive selection process, unwanted random integrations and/or eventual plasmidic-based concatemer multiple integrations at the expected locus can be rejected by exerting a counter selection process. The presence of a suicide gene marker out of the recombinogenic element can be circumvented by treating final selected cell clones by a prodrug that is dependent on the type of suicide gene marker used (i.e. ganciclovir for HSV TK and 5-fluorocytosine for CD:UPRT as examples). Whereas isogenic (monocopy) integrations are prodrug-resistant, all other types of integrants (random or concatemeric) are prodrug-sensitive.

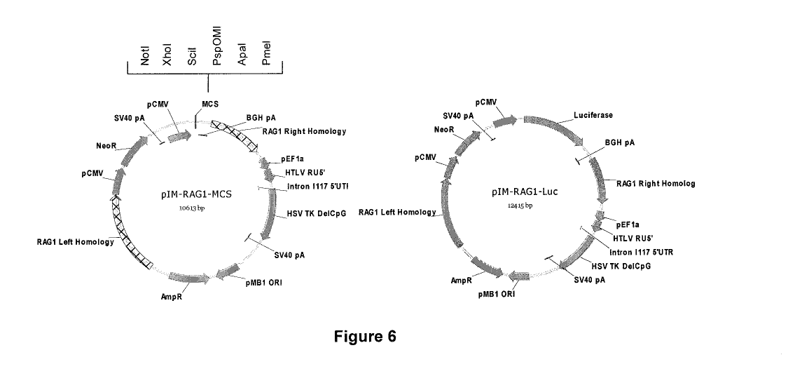

FIG. 6: Integration plasmid maps for targeting the human RAG1 locus. Left and right homology arms of the human RAG1 locus have been cloned into pIM-Universal-TK-Neo plasmid.

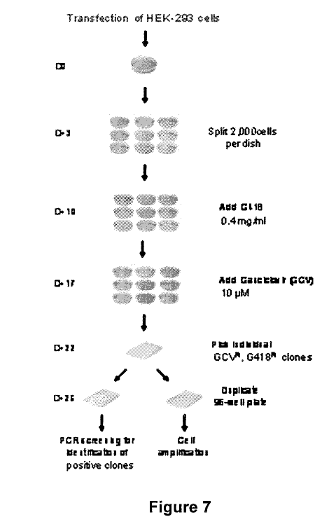

FIG. 7: Description of the selection process of targeted clones in HEK 293. HEK293 are transfected with the RAG1 meganuclease expression and the integration matrix. Three days post-transfection, 2,000 transfected cells are seeded in 10 cm culture dishes. Ten days post-transfection, neomycin-resistant clones are identified by culturing clones in the presence of G418 for 7 days. Seventeen days post-transfection, neomycin- and ganciclovir-resistant clones are isolated by adding ganciclovir for 5 days. At the end of this selection process, double resistant clones are re-arrayed in 96-well plates. 96-well plates of clones are duplicated in order to be screened by PCR.

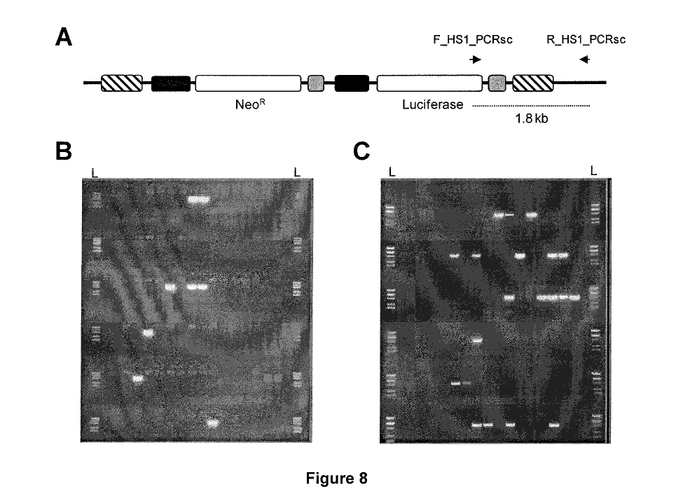

FIG. 8: Screen PCR of targeted clones in HEK293. A. Schematic representation of the RAG1 locus after targeted integration. PCR primer locations are depicted. B. and C. UV light pictures of ethidium bromide-stained, 96-well agarose gels, identifying PCR positive clones. 6 rows of 16 wells can be loaded per gel. On each side of each row, a DNA marker ladder (L) is loaded. DNA band sizes are (from top to bottom): 10 kb, 8 kb, 2 kb, 0.8 kb, 0.4 kb.

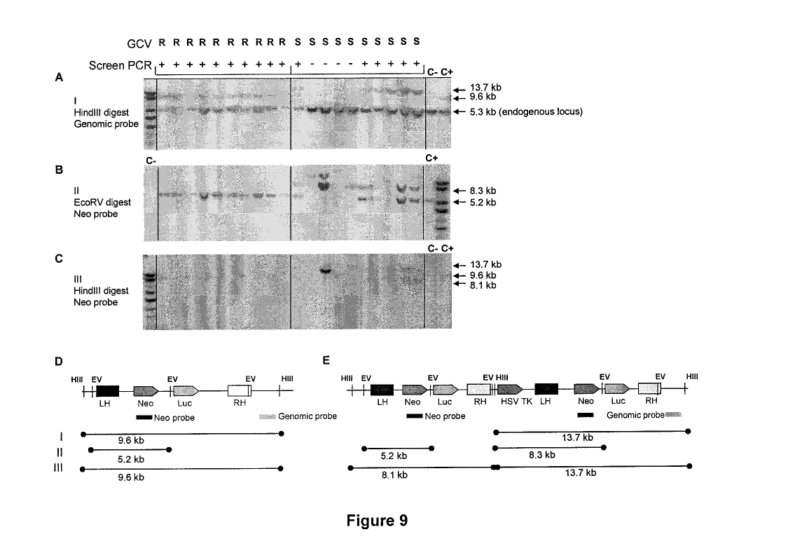

FIG. 9: Molecular characterization (Southern blot) of targeted clones in HEK293. A. Hybridization of the genomic probe on gDNA digested with HindIII restriction enzyme. B. Hybridization of the neomycin probe on gDNA digested with EcoRV restriction enzyme. C. Hybridization of the neomycin probe on gDNA digested with HindIII restriction enzyme. D. Schematic representation of the human RAG1 locus after monocopy targeted integration and expected band sizes. E. Schematic representation of the human RAG1 locus after multicopy targeted integration and expected band sizes. Abbreviations: GCV R; ganciclovir-resistant, GCV S; ganciclovir-sensitive, C−; untransfected HEK293 cells, C+; Positive targeted HEK293 clone, kb; kilobase, HIII; HindIII, EV; EcoRV, LH; left homology arm, RH; right homology arm, Neo; neomycin resistance gene, Luc; Luciferase reporter gene, HSV TK; herpes simplex virus thymidine kinase gene.

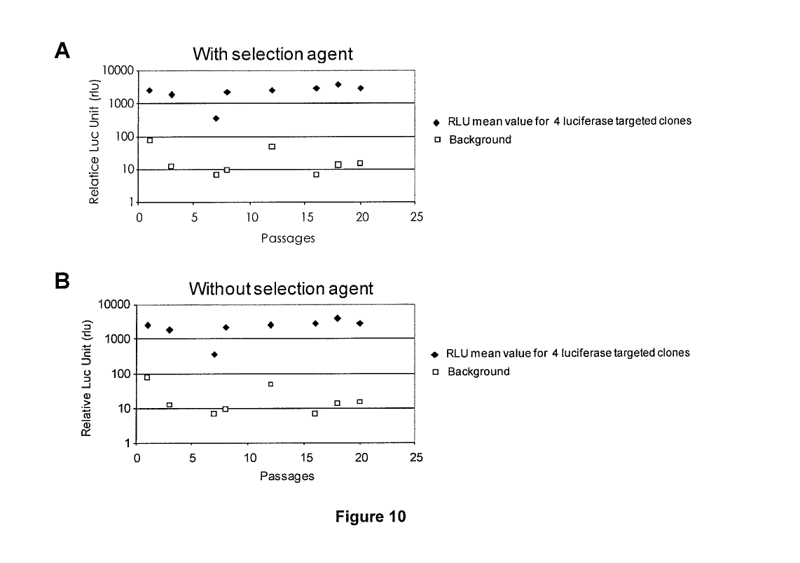

FIG. 10: Stability of the luciferase reporter gene expression in human RAG1-targeted HEK293 clones. A. Expression of luciferase (mean value for 4 luciferase targeted clones) over a period of 20 passages in the presence of the selection agent. B. Expression of luciferase (mean value for 4 luciferase targeted clones) over a period of 20 passages in the absence of the selection agent.

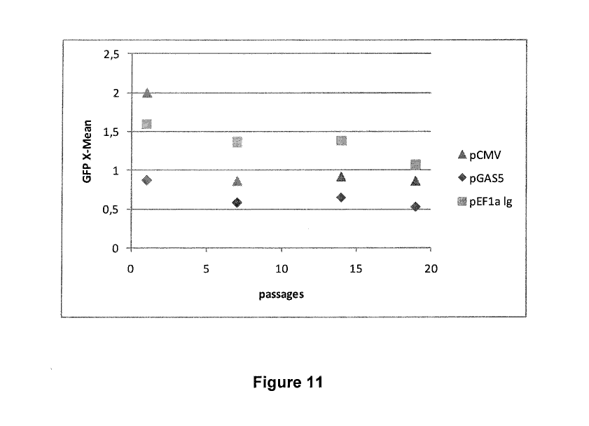

FIG. 11: Stability of TagGFP2 reporter gene under the control of three different promoters in human RAG1-targeted HEK293 clones. Expression of TagGFP2 (GFP X-mean) under the control of EFIa (square), CMV (triangle) or GAS5 (circle) promoters over a period of 20 passages.

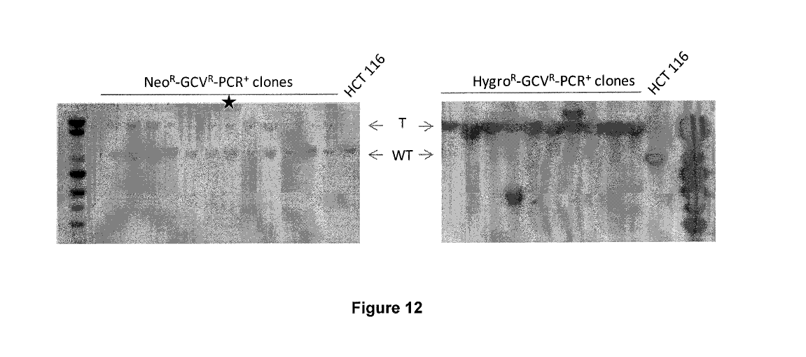

FIG. 12: Southern blot analysis of mono-allelic and bi-allelic RAG1 disrupted gene in targeted HCT 116 clones. Left panel: Hybridization of the genomic probe on gDNA digested with HindIII restriction enzyme from Neo^{R}GCV^{R}PCR⁺ clones. Control lane (gDNA from native HCT 116). Black star (D12 clone used for the second targeting experiment). Right panel: Hybridization of the genomic probe on gDNA digested with HindIII restriction enzyme from Hygro^{R}GCV^{R}PCR⁺ clones. T: targeted allele, WT: wild type allele.

There will now be described by way of example a specific mode contemplated by the Inventors. In the following description numerous specific details are set forth in order to provide a thorough understanding. It will be apparent however, to one skilled in the art, that the present invention may be practiced without limitation to these specific details. In other instances, well known methods and structures have not been described so as not to unnecessarily obscure the description.

### EXAMPLE 1

### Design of Meganuclease-Encoding Plasmid(s)

Several groups including the inventors have modified the recognition capability of meganucleases in order to target natural genomic DNA sequences of particular interest. These newly developed enzymes are designed according to meganucleases that exist in nature; the applicants have used them to target well-defined DNA sequences for a given application. The applicants have developed a high-throughput screening platform for meganucleases to create a vast collection of “DNA scissors” and associate them with modified-specificity technologies.

Concerning such engineered meganucleases with a modified specificity of recognition, the examples given in the herein presented invention concern protein modifications from the I-CreI original backbone. However, the present invention can be applied to any other meganuclease backbone, such as I-SceI, I-CreI, I-MsoI, PI-SceI, I-Anil, PI-PfuI, I-DmoI, I-CeuI, I-Tsp0611 or functional hybrid proteins such as the I-DmoI moiety fused with an I-CreI peptide.

Most meganuclease proteins are actually monomers, but they nevertheless conserve a dual internal symmetry, with two DNA-binding half-sites each interacting with one half of the target DNA. It is not the case for I-CreI-derived engineered meganucleases which are composed of two separate sub-units and do therefore form a heterodimeric composition with each sub-unit recognizing half-site of the recognition locus. The Applicants have already shown that the fusion of both monomers was possible, by linking them with a short peptide sequence, while maintaining the functional cleavage activity (i.e. with demonstrations been given from extra- and intra-chromosomal target sequences). From this initial paradigm and as represented in FIG. 2, the expression of I-CreI-derived engineered meganucleases can be made using:
By two separate DNA plasmids/sequences in the same plasmid from which each monomeric moiety is expressed;
From the same plasmid by using the single-chain version composed of the fusion of both monomeric moieties.

As in the case for integration matrices that contain other expression cassettes, cis-active DNA elements that drive the transcription of meganuclease open-reading frame(s) (i.e. promoting sequences and polyadenylation signals) can be changed depending upon the target cell line and the relative properties of such genetic elements therein.

### EXAMPLE 2

### Design of Integration Matrices

Universal plasmid backbones have been designed and constructed in order to allow meganuclease driven HR in any cell type (FIG. 3). Certain genetic elements which are cloned in the integration matrix are mandatory such as the homology arms, the selection cassette and the GOI expression cassette.

The homology arms are necessary to achieve specific gene targeting. They are produced by PCR amplification using specific primers for i) the genomic region upstream of the meganuclease target site (left homology arm) and ii) the genomic region downstream of the meganuclease target site (right homology arm). The length of the homology arms are comprised between 500 bp and 2 kb, usually 1.5 kb.

The positive selection cassette is composed of a resistance gene controlled by a promoter region and a terminator sequence, which is also the case for the counter (negative) selection cassette. Examples of plasmid maps for these type of genetic elements inserted in universal integration matrices [pIM-Universal-TK-Neo (SEQ ID NO 1), pIM-Universal-CD:UPRT-Hygro (SEQ ID NO 2)] are given in FIG. 4, where positive (neomycin or hygromycin) and negative (HSV TK or CD:UPRT) selection marker genes are indicated. A list of genes implicated for positive and counter (negative) selection is given in Table I and includes neomycin phosphotransferase resistant gene, nptl (SEQ ID NO 3), hygromycin phosphotransferase resistant gene, hph (SEQ ID NO 4), puromycin N-acetyl transferase gene, pac (SEQ ID NO 5), blasticidin S deaminase resistant gene, bsr (SEQ ID NO 6), bleomycin resistant gene, sh ble (SEQ ID NO 7), Thymidine kinase gene of the herpes simplex virus deleted of CpG islands, HSV TK DelCpG (SEQ ID NO 8), cytosine deaminase coupled to uracyl phosphoribosyl transferase gene deleted of CpG islands, CD:UPRT DelCpG (SEQ ID NO 9).

The expression cassette is composed of a multiple cloning site (MCS) where the GOI is cloned using classical molecular biology techniques. The MCS is flanked by promoter (upstream) and terminator (downstream) sequences. The list of such genetic elements is given in Table II and includes cytomegalovirus immediate-early promoter, pCMV (SEQ ID NO 10), simian virus 40 promoter, pSV40 (SEQ ID NO 11), human elongation factor 1α promoter, phEF1α (SEQ ID NO 12), human phosphoglycerate kinase promoter, phPGK (SEQ ID NO 13), murine phosphoglycerate kinase promoter, pmPGK (SEQ ID NO 14), human polyubiquitin promoter, phUbc (SEQ ID NO 15), thymidine kinase promoter from human herpes simplex virus, pHSV-TK (SEQ ID NO 16), human growth arrest specific 5 promoter, phGAS5 (SEQ ID NO 17), tetracycline-responsive element, pTRE (SEQ ID N018), internal ribosomal entry site (IRES) sequence from encephalopathy myocarditis virus, IRES EMCV (SEQ ID NO 19), IRES sequence from foot and mouth disease virus, IRES FMDV (SEQ ID NO 20), SV40 polyadenylation signal, SV40 pA (SEQ ID NO 21), bovine growth hormone polyadenylation signal, BGH pA (SEQ ID NO 22).

From this basic scaffold, numerous integration matrices could be derived. For instance, a double MCS separated by an IRES sequence can be introduced to express two GOIs. The MCS can be equipped with in frame short sequences (N-term or C-term) allowing the tagging of GOIs. Multiple applications can then be envisioned according to the type of tag that is attached (imaging, purification, immunodetection, cellular addressing).

Table III gives an overview of optional genetic elements that can be introduced in the integration vector, including FLAG (SEQ ID NO 23), FLASH/REASH (SEQ ID NO 24), IQ (SEQ ID NO 25), histidine (SEQ ID NO 26), STREP (SEQ ID NO 27), streptavidin binding protein, SBP (SEQ ID NO 28), calmodulin binding protein, CBP (SEQ ID NO 29), haemagglutinin, HA (SEQ ID NO 30), c-myc (SEQ ID NO 31), V5 tag sequence (SEQ ID NO 32), nuclear localization signal (NLS) from nucleoplasmin (SEQ ID NO 33), NLS from SV40 (SEQ ID NO 34), NLS consensus (SEQ ID NO 35), thrombin cleavage site (SEQ ID NO 36), P2A cleavage site (SEQ ID NO 37), T2A cleavage site (SEQ ID NO 38), E2A cleavage site (SEQ ID NO 39).

In addition, reporter genes, from which a list is given in Table IV, can also be cloned into the MCS and can serve as positive controls for evaluating the expression level after targeted integration at the expected chromosomal locus. These include firefly luciferase gene (SEQ ID NO 40), renilla luciferase gene (SEQ ID NO 41), β-galactosidase gene, LacZ (SEQ ID NO 42), human secreted alkaline phosphatase gene, hSEAP (SEQ ID NO 43), murine secreted alkaline phosphatase gene, mSEAP (SEQ ID NO 44).

Finally, meganuclease-induced targeted integration can be sometimes accompanied with unwanted events such as random insertion of the integration matrix in the host genome. Usually, this phenomenon involved the complete insertion of the integration matrix including sequences of the plasmid backbone. In order to avoid, at least partially this phenomenon, the presence of a counter (negative) selection marker is present in the backbone part of the plasmid (i.e. outside the homology arms) as described for instance in Khanahmad et al, 2006 and Jin et al, 2003.

The use of a this type of suicide gene expression system in the context of meganuclease-driven targeted integration is particularly relevant for eliminating targeted cell clones that are associated with potential random insertions.

In cellulo linearization of the integration matrix can also lead to random integration in the host genome. If the linearization occurs within the negative marker and then inactivates its function, those random integration events would not be eliminated by the pro-drug treatment of cells. In order to circumvent this drawback, the inventors propose an integration matrix comprising the presence of two negative selection expression cassettes on the integration matrix; for instance one upstream of the HOMO1 region and one downstream of the HOMO2 region. The inventors have shown that the use of at least one negative selection expression cassettes prevents from multicopy-targeted integrations. Previous uses of counter negative selection marker were described for preventing from random integration. The inventors have now shown that these markers allow also for the prevention of multicopy-targeted integrations.

Integration matrices that contain a suicide gene expression cassette in the plasmidic backbone out of the recombinogenic element allow the selection of targeted cell clones with enrichment of integration events at the expected chromosomal locus. The maintenance of the suicide gene expression cassette in some of targeted cell clones is an unwanted integration event since the exact targeted process normally rejects the integration of plasmid-based sequences which are located out of the recombinogenic element. By treating cell clones with the toxic prodrug related to the suicide gene system, it is therefore possible to kill the ones that contain such type of integrants (FIG. 5).

The present invention for targeted integration at a given chromosomal locus can also be derived by using integration matrices from other types of DNA origin than the classic plasmid-based system. These include any type of viral vectors wherein DNA intermediates are generated, such as non-integrative retroviruses and lentiviruses by taking advantage of their 1 LTR and 2LTR circular proviruses, episomal DNA viral vectors including adenoviruses and adeno-associated viruses, as well as other types of DNA viruses having an episomal replicative status.

### EXAMPLE 3

### Transfection and Selection

In this example, we present the technical process leading to the identification of GOI targeted integration, using a meganuclease specific for a target located in the RAG1 human gene. Plasmid maps related to RAG1-specific integration matrices that have been used for the demonstrations given here below [pIM-RAG1-MCS (SEQ ID NO 45) pIM-RAG1-Luc (SEQ ID NO 46)] are depicted in FIG. 6. Since the engineered meganuclease can recognize and cut within the human RAG1 gene, targeted integration can be obtained in virtually all human cell lines. Depending of the capacity of cells to adhere to plastic, transfection and selection procedures are different but both lead to the efficient identification of targeted cell clones.

Integration matrix and meganuclease expression vector are transfected into cells using known techniques. There are various methods of introducing foreign DNA into a eukaryotic cell and many materials have been used as carriers for transfection, which can be divided into three kinds: (cationic) polymers, liposomes and nanoparticles. Other methods of transfection include nucleofection, electroporation (for instance Cyto Pulse (Cellectis)), heat shock, magnetofection and proprietary transfection reagents such as Lipofectamine, Dojindo Hilymax, Fugene, JetPEI, Effectene, DreamFect, PolyFect, Nucleofector, Lyovec, Attractene, Transfast, Optifect.

### 3.1 Transfection and Selection of Adherent HEK-293 Cells

Here is described, as an example, the procedure used for the transfection of HEK-293 (human adherent cell line) with Lipofectamine® (FIG. 7).

### Materials and Methods

One day prior to transfection, HEK-293 cells are seeded in a 10 cm tissue culture dish (10⁶ cells per dish). On transfection day (D), Human RAG1 meganuclease expression plasmid and integration matrix (pIM-RAG1-MCS (SEQ ID NO 45) and its derived GOI-containing plasmid with the GOI in place of the MCS, or pIM-RAG1-Luc (SEQ ID NO 46) as positive control) are diluted in 300 μl of serum-free medium. On the other hand, 10 μl of Lipofectamine® reagent is diluted in 290 μl μl of serum-free medium. Both mixes are incubated 5 minutes at room temperature. Then, the diluted DNA is added to the diluted Lipofectamine® reagent (and never the way around). The mix is gently homogenized by tube inversion and incubated 20 minutes at room temperature. The transfection mix is then dispensed over plated cells and transfected cells are incubated in a 37° C., 5% CO₂ humidified incubator. The next day, transfection medium is replaced with fresh complete medium.

Three days after transfection, cells are harvested and counted. Cells are then seeded in 10 cm tissue culture dishes at the density of 200 cells/ml in a total volume of 10 ml of complete medium. 10 cm tissue culture dishes are incubated at 37° C., 5% CO₂ for a total period of 7 days. At the end of the 7 days, single colonies of cells are visible.

Ten days after transfection (or seven days after plating), culture medium is replaced with fresh medium supplemented with selection agent (i.e. corresponding to the resistance gene present on the integration matrix). In this example, the integration matrix contains a full neomycin resistance gene (FIG. 6). Therefore, selection of clones is done with G418 sulfate at the concentration of 0.4 mg/ml. The medium replacement is done every two or three days for a total period of seven days. At the end of this selection phase, resistant cells can be either isolated in a 96-well plates or maintained in the 10 cm dish (adherent cells) or re-arrayed in new 96-well plates (suspension cells) for counter selection.

Since the HSV TK counter selection marker is present on the integration matrix (FIG. 6), resistant cells or colonies can be cultivated in the presence of 10 μM of ganciclovir (GCV) to eliminate unwanted integration events such as random insertion and multicopy-targeted integrations. After 5 days of culture in the presence of GCV, double resistant (G418^{R}-GCV^{R}) cell colonies can be isolated for further characterization.

At the end of this selection phase, resistant (G418^{R}-GCV^{R}) cell colonies can be isolated for molecular screening by PCR (see §3.8).

### 3.2 Transfection and Selection of Adherent U-2 OS Cells

Here is described, as an example, the procedure used for the transfection of U-2 OS (human adherent cell line) with the Amaxa® Cell Line Nucleofector® Kit V reagents (Lonza).

### Materials and Methods

On transfection day (D), cells should not be more than 80% confluent. Cells are harvested from their sub-culturing vessel (T162 Tissue Culture Flask) by trypsinization and are collected in a 15 ml conical tube. Harvested cells are counted. 10⁶ cells are needed per transfection point. Cells are centrifuged at 300 g for 5 min and resuspended in Cell Line Nucleofector® Solution V at the concentration of 10⁶cells/100 μl. Amaxa electroporation cuvette is prepared by adding i) the hsRAG1 Integration Matrix CMV Neo (pIM.RAG1.CMV.Neo SEQ ID NO: 58) containing the gene of interest, or the hsRAG1 Integration Matrix CMV Neo Luc (pIM.RAG1.CMV.Neo.Luc SEQ ID NO: 59) and the hsRAG1 Meganuclease Plasmids (SEQ ID NO: 60) ((Endofree quality preparation), ii) 100 μl of cell suspension (10⁶ cells). Cells and DNA are gently mixed and electroporated using Amaxa® program X-001. Immediately after electroporation, pre-warmed complete medium is added to cells and cells suspension is split into two 10 cm dishes (5 ml per dish) containing 5 ml of 37° C. pre-warmed complete medium. 10 cm dishes are then incubated in a 37° C., 5% CO₂ humidified incubator.

Two days after transfection (D+2) the complete culture medium is replaced with fresh complete medium supplemented with 0.4 mg/ml of G418. This step is repeated every 2 or 3 days for a total period of 7 days. At D+9, the complete culture medium supplemented with 0.4 mg/ml G418 is replaced with fresh complete medium supplemented with 0.4 mg/ml of G418 and 50 μM Ganciclovir. This step is repeated every 2 or 3 days for a total period of 5 days. At D+14, G418 and GCV resistant clones are picked in a 96-well plate. At this step cells are maintained in complete medium supplemented with 0.4 mg/ml of G418 only.

At the end of this selection phase, resistant (G418^{R}-GCV^{R}) cell colonies can be isolated for molecular screening by PCR (see §3.8).

### 3.3 Transfection and Selection of Adherent HCT116 Cells

Here is described, as an example, the procedure used for the transfection of HCT 116 (human adherent cell line) with FuGENE® HD (Promega).

### Materials and Methods

One day prior to transfection, HCT 116 cells are seeded in a 10 cm tissue culture dish (5×10⁵ cells per dish). On transfection day (D), Human RAG1 meganuclease expression plasmid and integration matrix (pIM-RAG1-MCS (SEQ ID NO 45) and its derived GOI-containing plasmid with the GOI in place of the MCS, or pIM-RAG1-Luc (SEQ ID NO 46) as positive control) are diluted in 500 μl of serum-free medium. Then, 15 μl of FuGENE® HD reagent is diluted in the DNA mix. The mix is gently homogenized by tube inversion and incubated 15 minutes at room temperature. The transfection mix is then dispensed over plated cells and transfected cells are incubated in a 37° C., 5% CO₂ humidified incubator.

The day after transfection (D+1) the complete culture medium is replaced with fresh complete medium supplemented with 0.4 mg/ml of G418. This step is repeated every 2 or 3 days for a total period of 7 days. At D+9, the complete culture medium supplemented with 0.4 mg/ml G418 is replaced with fresh complete medium supplemented with 0.4 mg/ml of G418 and 50 μM Ganciclovir. This step is repeated every 2 or 3 days for a total period of 5 days. At D+14, G418 and GCV resistant clones are picked in a 96-well plate. At this step cells are maintained in complete medium supplemented with 0.4 mg/ml of G418 only.

At the end of this selection phase, resistant (G418^{R}-GCV^{R}) cell colonies can be isolated for molecular screening by PCR (see §3.8).

3.4 Transfection and Selection of Adherent HepG2 cells

Here is described, as an example, the procedure used for the transfection of HepG2 (human adherent cell line) with FuGENE® HD.

### Materials and Methods

One day prior to transfection, HCT 116 cells are seeded in a 10 cm tissue culture dish (10⁶ cells per dish). On transfection day (D), Human RAG1 meganuclease expression plasmid and integration matrix (pIM-RAG1-MCS (SEQ ID NO: 45) and its derived GOI-containing plasmid with the GOI in place of the MCS, or pIM-RAG1-Luc (SEQ ID NO: 46) as positive control) are diluted in 500 μl of serum-free medium. Then, 15 μl of FuGENE® HD reagent is diluted in the DNA mix. The mix is gently homogenized by tube inversion and incubated 15 minutes at room temperature. The transfection mix is then dispensed over plated cells and transfected cells are incubated in a 37° C., 5% CO₂ humidified incubator.

Three days after transfection (D+3), transfected cells are harvested by trypsinization and split into two 10 cm dishes. The complete culture medium is replaced with fresh complete medium supplemented with 0.8 mg/ml of G418. This step is repeated every 3 days for a total period of 10 days. At D+13, the complete culture medium supplemented with 0.8 mg/ml G418 is replaced with fresh complete medium supplemented with 0.8 mg/ml of G418 and 50 μM Ganciclovir. This step is repeated every 2 or 3 days for a total period of 5 days. At D+18, cells are cultivated in fresh complete medium supplemented with 0.8 mg/ml of G418. At D+24, G418 and GCV resistant clones are picked in a 96-well plate. At this step cells are maintained in complete medium supplemented with 0.8 mg/ml of G418 only.

At the end of this selection phase, resistant (G418^{R}-GCV^{R}) cell colonies can be isolated for molecular screening by PCR (see §3.8).

3.5 Transfection and Selection of Adherent MRC-5 Cells

Here is described, as an example, the procedure used for the transfection of MRC-5 (human adherent cell line) with PolyFect® (Qiagen).

### Materials and Methods

One day prior to transfection, MRC-5 cells are seeded in a 10 cm tissue culture dish (2.5×10⁵ cells per dish). On transfection day (D), Human RAG1 meganuclease expression plasmid and integration matrix (pIM-RAG1-MCS (SEQ ID NO 45) and its derived GOI-containing plasmid with the GOI in place of the MCS, or pIM-RAG1-Luc (SEQ ID NO 46) as positive control) are diluted in 275 μl of serum-free medium. Then, 50 μl of PolyFect® HD reagent is diluted in the DNA mix. The mix is gently homogenized by tube inversion and incubated 10 minutes at room temperature. 700 μl of complete medium is added to the transfection mix and the final mix is then dispensed over plated cells and transfected cells are incubated in a 37° C., 5% CO₂ humidified incubator.

Three days after transfection, cells are harvested and counted. Cells are then seeded in 10 cm tissue culture dishes at the density of 1000 cells/ml in a total volume of 10 ml of complete medium. 10 cm tissue culture dishes are incubated at 37° C., 5% CO₂ for a total period of 7 days. At the end of the 7 days, single colonies of cells are visible. Ten days after transfection (or seven days after plating), culture medium is replaced with fresh medium supplemented with G418 sulfate at the concentration of 0.4 mg/ml. The medium replacement is done every two or three days for a total period of seven days. At D+13, the complete culture medium supplemented with 0.4 mg/ml G418 is replaced with fresh complete medium supplemented with 0.4 mg/ml of G418 and 50 μM Ganciclovir. This step is repeated every 2 or 3 days for a total period of 5 days.

At the end of this selection phase, resistant (G418^{R}-GCV^{R}) cell colonies can be isolated for molecular screening by PCR (see §3.8).

3.6 Transfection and Selection of Suspension Jurkat Cells

Here is described, as an example, the procedure used for transfection of Jurkat cells (human lymphoblastoid cell line) with the Amaxa® Cell Line Nucleofector® Kit V(Lonza).

### Materials and Methods

On transfection day (D), Jurkat cells are collected in a 15 ml conical tube and counted. 2×10⁶ cells are needed per transfection point. Cells are centrifuged at 300 g for 5 min and resuspended in Cell Line Nucleofector® Solution V at the concentration of 2×10⁶cells/1004 Amaxa electroporation cuvette is prepared by adding i) the hsRAG1 Integration Matrix CMV Neo (pIM.RAG1.CMV.Neo SEQ ID NO: 58) containing the gene of interest, or the hsRAG1 Integration Matrix CMV Neo Luc (pIM.RAG1.CMV.Neo.Luc SEQ ID NO: 59) and the hsRAG1 Meganuclease Plasmid (SEQ ID NO: 60) ((Endofree quality preparation), ii) 100 μl of cell suspension (2×10⁶ cells). Cells and DNA are gently mixed and electroporated using Amaxa® program X-001. Immediately after electroporation, pre-warmed complete medium is added to cells and cells suspension is transferred into a well of a 6 well plate containing 2.4 ml of pre-warmed complete medium. 6 well plates are then incubated in a 37° C., 5% CO₂ humidified incubator.

Three days after transfection (D+2) the complete culture medium is replaced with fresh complete medium supplemented with 0.7 mg/ml of G418. This step is repeated every 2 or 3 days for a total period of 17 days. After this selection period, resistant cells are harvested and cloned in round-bottom 96 well plates at the cells/well density in complete medium supplemented with 0.7 mg/ml of G418. After sufficient growth (10-15 days), resistant (G418^{R}) cell clones can be isolated for molecular screening by PCR (see §3.8).

In the case of Jurkat cells, the counter selection process (Ganciclovir) is not applied since the Jurkat cell line is extremely sensitive to the drug even at very low concentration.

3.7 Transfection and Selection of Suspension K-562 Cells

Here is described, as an example, the procedure used for transfection of K-562 cells (human lymphoblastoid cell line) with the Amaxa® Cell Line Nucleofector® Kit V(Lonza).

### Materials and Methods

On transfection day (D), K-562 cells are collected in a 15 ml conical tube and counted. 10⁶ cells are needed per transfection point. Cells are centrifuged at 300 g for 5 min and resuspended in Cell Line Nucleofector® Solution V at the concentration of 10⁶cells/100 μl. Amaxa electroporation cuvette is prepared by adding i) the hsRAG1 Integration Matrix CMV Neo (pIM.RAG1.CMV.Neo SEQ ID NO: 58) containing the gene of interest, or the hsRAG1 Integration Matrix CMV Neo Luc (pIM.RAG1.CMV.Neo.Luc SEQ ID NO: 59) and the hsRAG1 Meganuclease Plasmids (SEQ ID NO: 60) ((Endofree quality preparation), ii) 100 μl of cell suspension (10⁶ cells). Cells and DNA are gently mixed and electroporated using Amaxa® program X-001. Immediately after electroporation, pre-warmed complete medium is added to cells and cells suspension is transferred into a well of a 6 well plate containing 2.4 ml of pre-warmed complete medium. 6 well plates are then incubated in a 37° C., 5% CO₂ humidified incubator.

Three days after transfection (D+3) the complete culture medium is replaced with fresh complete medium supplemented with 0.5 mg/ml of G418. This step is repeated every 2 or 3 days for a total period of 7 days. At D+10, the complete culture medium supplemented with 0.4 mg/ml G418 is replaced with fresh complete medium supplemented with 0.5 mg/ml of G418 and 50 μM Ganciclovir. This step is repeated every 2 or 3 days for a total period of 5 days.

After this selection period, resistant cells are harvested and cloned in round-bottom 96 well plates at the 10 cells/well density in complete medium supplemented with 0.5 mg/ml of G418. After sufficient growth (10-15 days), resistant (G418^{R}-GCV^{R}) cell clones can be isolated for molecular screening by PCR (see §3.8).

### 3.8 PCR Screening

Once the selection and optionally counter selection is achieved, resistant colonies or clones, re-arrayed in 96-well plates are maintained in the 96-well format. Replicas of plates are done in order to generate genomic DNA from resistant cells. PCR are then performed to identify targeted integration.

### Materials and Methods

Genomic DNA preparation: genomic DNAs (gDNAs) from double resistant cell clones are prepared with the ZR-96 Genomic DNA Kit™ (Zymo Research) according to the manufacturer's recommendations.

PCR Primer Design:

In the present example (human RAG1 locus), PCR primers are chosen according to the following rules and as represented in panel A of FIG. 8. The forward primer is located in the heterologous sequence (i.e. between the homology arms). For instance the forward PCR primer is situated in the BGH polyA sequence (SEQ IN NO 22), terminating the transcription of the GOI. The reverse PCR primer is located within the RAG1 locus but outside the right homology arm. Therefore, PCR amplification is possible only when a specific targeted integration occurs. Moreover, this combination of primers can be used for the screening of targeted events, independently to the GOI to be integrated.

| (SEQ ID NO: 47) | |
|---|---|
| F_HS1_PCR_{SC}: | GGAGGATTGGGAAGACAATAGC |
| (SEQ ID NO: 48) | |
| R_HS1_PCR_{SC}: | CTTTCACAGTCCTGTACATCTTGT |

PCR Conditions:

PCR reactions are carried out on 5 μl of gDNA in 25 μl final volume with 0.25 μM of each primers, 10 μM of dNTP and 0.5 μl of Herculase II FusionDNA polymerase (Stratagene).

PCR Program:

| Temperature | Time | Cycle |
|---|---|---|
| (° C.) | (minutes) | number |
| 95 | 5 | 1 |
| 95 | 1 | 30 |
| 55 | 1 | |
| 72 | 1.5 | |
| 72 | 10 | 1 |

### Results

An example of the PCR screening process for targeted events in the human RAG1 system is presented in FIG. 8. On panel A, a schematic representation of the RAG1 locus after targeted integration is shown with the location of the screening PCR primers and the expected band size. On panels B and C, are shown the results of the PCR screening on gDNA from G418^{R}-GCV^{R} targeted cell clones that have been obtained through the process described above. The double resistant clones have been re-arrayed in 96-well plates. After few days in culture, 96-well plates are duplicated and one of the replicas is used for gDNA preparation, while the other parallel 96-well plate is kept in culture. gDNA is submitted to the PCR amplification and 10 μl of PCR reaction are loaded on a 0.8% agarose gel and submitted to electrophoresis. After migration, the gel is stained with ethidium bromide and exposed to UV light in order to identify PCR positive clones. On panel B, we identified 8 clones out of 96 where a specific DNA band shows up, which represents a success rate of 8.3%. On panel C, 20 clones out of 96, representing a success rate of 20.8%, are identified.

According to this molecular screening by PCR, results of targeted integration into the hsRAG1 locus of the different human cell lines, for which a specific protocol has been developed (see §3.1 to 3.7) are summarized in Table V. The level of specific targeted integration is comprised between 7% and 44%, demonstrating the efficacy of the cGPS custom system. It demonstrates that the present invention could be applied to any kind of cell lines (adherent, suspension, primary cell lines).

TABLE V
Summary of targeted integration in the different cell lines.

| | | Targeted | Single copy |
|---|---|---|---|
| | | clones (%) | integrants (%) |
| Adherent | HEK-293 | 44 | 71 |
| cell line | U-2 OS | 16 | 85 |
| | HCT 116 | 7 | 70 |
| | HepG2 | 15 | 69 |
| | MRC-5 | 7 | 59 |
| Suspension | Jurkat | 13 | 90 |
| cell line | K-562 | 11 | 82 |

In order to further characterize these positive clones, cells from corresponding wells, maintained in culture are individually amplified from the 96-well plate format to a 10 cm dish culture format.

### 3.9 Molecular Characterization (Southern Blot)

A correct targeted insertion in double resistant clones can be easily identified at the molecular level by Southern blot analysis (FIG. 9).

### Materials and Methods

gDNA from targeted clones was purified from 10⁷ cells (about a nearly confluent 10 cm dish) using the Blood and Cell culture DNA midi kit (Qiagen). 5 to 10 μg of gDNA are digested with a 10-fold excess of restriction enzyme by overnight incubation (here HindIII or EcoRV restriction enzymes). Digested gDNA is separated on a 0.8% agarose gel and transfer on nylon membrane. Nylon membranes are then probed with a ³²P DNA probe specific either for the neomycin gene or for a RAG1 specific sequence located outside the 3′ homology arm (panels D and E of FIG. 9). After appropriate washes, the specific hybridization of the probe is revealed by autoradiography (panels A to C of FIG. 9).

### Results

In the example presented here, we compared the hybridization patterns for G418^{R} clones with different phenotypes (indicated on the top of panel A). From G418^{R}-PCR⁺ cell clones, 10 GCV^{R} and 6 GCV^{S} targeted cell clones have been analyzed, and 4 G418^{R} cell clones from the G418^{R}-PCR⁻ phenotype have also been characterized by Southern blotting. gDNA from these clones have been digested with HindIII restriction enzyme (panels A and C) or EcoRV (panel B) and hybridized with the RAG1 genomic probe (panel A) or with the neomycin probe (panel B and C). Schematic representation of the RAG1 targeted locus and expected band size according to the restriction enzyme digest and the probe used are depicted on panel D. All G418^{R}-GCV^{R}-PCR⁺ clones show a molecular genetic pattern conform to the initial prediction of isogenic (monocopy) integration. On panel A, since we used a RAG1 genomic probe, we revealed another band at 5.2 kb that corresponds to one of the RAG1 allele that has not been targeted. This band is also present on the negative control (C−: untransfected HEK293 cells) and G418^{R}-GCV^{s}-PCR positive and negative clones. These results demonstrate that for all G418^{R}-GCV^{R}-PCR⁺ clones, one allele of the human RAG1 locus has been targeted through meganuclease induced homologous recombination.

By contrast, G418^{R}-GCV^{s}-PCR⁻ clones do not show any specific bands indicative of a targeted event. Although specific bands are obtained with the neomycin probe, their sizes do not match with the expected size. These clones come from the random integration of the integration matrix in the host genome. The use of the counter selection marker such as HSV TK with its GCV active prodrug allows the elimination of such unwanted events.

In addition, G418^{R}-GCV^{s}-PCR⁺ clones show a genetic pattern slightly different to G418^{R}-GCV^{R}-PCR⁺ positive clones. Indeed, G418^{R}-GCV^{s}-PCR⁺ positive clones show a pattern that is compatible with a multicopy targeted integration that is depicted on panel E. The multicopy targeted integration involved the integration of the HSV TK gene (from plasmid DNA backbone of the integration matrix) and therefore renders cells sensitive to GCV.

All the data presented in this example demonstrate that the use of custom meganuclease induced gene targeting technique combined with a robust selection process leads to efficient identification of targeted event. Such targeted events could be either monocopy- or multicopy-targeted integrations that can be discriminated via a robust counter selection process that has been developed. In a similar way, this counter selection process also allows to reject cell clones having random-associated integrations in their chromosomes.

### EXAMPLE 4

### GOI Expression and Stability

### 4.1 Luciferase Expression

In this example, the inventors monitored the level of expression of four targeted clones expressing the luciferase gene. The firefly luciferase reporter gene (SEQ ID NO 40) has been cloned in pIM-RAG1-MCS (SEQ ID NO 45). The resulting vector (pIM-RAG1-Luc, SEQ ID NO 46) has been transfected in HEK293 cells according to the protocol described in example 3. Targeted cell clones surviving the selection and counter selection processes described in example 3 are isolated and characterized according to section §3.7 and §3.8.

The 4 HEK293 luciferase-targeted clones were maintained in culture over a period of 20 passages (two passages per week). Each clone was cultured in the presence of selection drug (G418: 0.4 mg/ml). Furthermore, the inventors evaluated the expression of the reporter gene for the same clones but without selection drug (i.e. in complete DMEM medium) over a period of time corresponding to 20 passages.

### Materials and Methods

Luciferase Expression:

Cells from targeted clones are washed twice in PBS then incubated with 5 ml of trypsin-EDTA solution. After 5 min. incubation at 37° C., cells are collected in a 15 ml conical tube and counted.

Cells are then resuspended in complete DMEM medium at the density of 50,000 cells/ml. 100 μl (5,000 cells) are aliquoted in triplicate in a white 96-well plate (Perkin-Elmer). 100 μl of One-Glo reagent (Promega) is added per well and after a short incubation the plate can be read on a microplate luminometer (Viktor, Perkin-Elmer).

### Results

The data are presented in FIG. 10. On panels A and B, the mean level of luciferase expression for 4 luciferase targeted clones is shown as a function of time in the presence or absence of selection agent, respectively. These data indicates that expression of the luciferase reporter gene is remarkably stable even after a long period of culture. Furthermore the presence of the selection agent is not necessary to ensure a long lasting expression of transgene since the stability of reporter expression is equivalent when the targeted clones are cultivated without selection agent.

### 4.2 GFP Expression and Stability

In this example, the inventors monitored the level of expression of targeted clones expressing the Green fluorescent Protein gene from Aequorea macrodactyla (TagGFP2 Evrogen SEQ ID NO 49). The TagGFP2 reporter gene (SEQ ID NO 49) has been cloned in the pIM-RAG1-MCS (SEQ ID NO 45), the pIM.RAG1.EFIa.MCS (SEQ ID NO 50) and the pIM.RAG1.GAS5.MCS (SEQ ID NO 51). The resulting vectors (pIM-RAG1-TagGFP2, SEQ ID NO 52, pIM.RAG1.EF1a.TagGFP2, SEQ ID NO 53 and pIM.RAG1.GAS5.TagGFP2, SEQ ID NO 54) have been transfected in HEK293 cells according to the protocol described in example 3.1. Targeted cell clones surviving the selection and counter selection processes described in example 3 are isolated and characterized according to section §3.7 and §3.8.

One HEK293 TagGFP2-targeted clone from each of the 3 constructs were maintained in culture over a period of 20 passages (two passages per week). Each clone was cultured in the absence of selection drug (G418) since it has been shown that the selection pressure was not necessary to maintain expression (see §4.1)

### Materials and Methods

Tag2GFP Expression:

Cells from targeted clones are washed twice in PBS then incubated with 5 ml of trypsin-EDTA solution. After 5 min. incubation at 37° C., cells are collected in a 15 ml conical tube and counted.

Cells are then resuspended in complete DMEM medium at the density of 50,000 cells/ml. Cell samples are then analyzed by flow cytometry using a MACSQuant device (Miltenyi Biotec). Fluorescence is collected using the green channel and expressed as the mean fluorescence unit.

### Results

The data are presented in FIG. 11. The mean fluorescence level of TagGFP expression for 3 different TagGFP2 targeted clones is shown as a function of time. These data indicates that expression of the TagGFP2 reporter gene under the control of 3 different promoters is remarkably stable even after a long period of culture (10 weeks) even in the absence of selection agent.

According to the promoter sequence, the mean level of fluorescence is variable. EF1a promoter gives the strongest TagGFP2 expression while GAS5 promoters gives weaker expression. The results indicate that the TagGFP2 expression can be modulated by the use of different promoters.

### 4.3 Fusion Protein Expression

### EXAMPLE 5

### Gene Inactivation (Knock Out) Through Targeted Integration

In this example, the inventors show evidence that the RAG1 locus has been disrupted by the sequential hs RAG1 meganuclease-driven targeted integration of i) a RAG 1 integration matrix bearing the neomycine resistance gene (pIM-RAG1-Luc, SEQ ID NO 46) and ii) a RAG1 integration matrix bearing the hygromycin resistance gene (pIM-RAG1-Hygro, SEQ ID NO 55).

### Materials and Methods

HCT 116 cells were transfected according to the protocol described in section §3.3. Neo^{R}-GCV^{R} resistant clones were screened by PCR described in section §3.8. Neo^{R}-GCV^{R}-PCR⁺ clones were analyzed by Southern Blot (see section §3.9). Among the identified targeted clones on one of the RAG1 allele, one clone (D12) has been selected and amplified. A second targeted experiment has been performed on this clone as described on section §3.3 except that the RAG1 integration matrix bearing the hygromycin resistance gene (SEQ ID NO 55) has been used. As a consequence, selection of clones has been based on hygromycin (0.6 mg/ml) instead of neomycin. Hygro^{R} clones have screened by PCR and PCR positive clones have analyzed by Southern Blot as described in sections §3.8 and §3.9.

### Results

On the left panel of FIG. 12 is presented the hybridization pattern for neo^{R}-GCV^{R}-PCR⁺ clones obtained after the first targeted experiment. The hybridization is performed with a genomic probe (see FIG. 9) after HindIII digest of genomic DNA. As shown in the control lane (HCT 116), untargeted RAG1 locus is identified by a 5.2 kb band. This band is present in all the targeted clones in addition to a second band (9.6 kb) indicated that one allele of the RAG1 gene is targeted (T) whereas the other allele is wild type (WT). One of these clones (clone D12, marked with a black star) has been used for the second experiment, aiming at targeting the second RAG1 allele. The hybridization pattern is shown on the right panel of FIG. 12. Again, the hybridization is performed with a genomic probe (see FIG. 9) after HindIII digest of genomic DNA. The 5.2 kb WT band is no more visible in all targeted clones. Instead, a unique band at 9.6 kb, specific for the targeted integration of heterologous sequences present in the integration matrices is observed in all clones but two. These results demonstrate that the RAG1 alleles have both been disrupted leading to the full inactivation of the RAG1 gene. This sequential approach for gene inactivation can be applied to other loci using other meganucleases.

### REFERENCES

Capecchi (2001) Generating mice with targeted mutations. Nat Med, 7, 1086-90.
Chevalier and Stoddard (2001) Homing endonucleases: structural and functional insight into the catalysts of intron/intein mobility. Nucleic Acids Res, 29, 3757-74.
Choulika, Perrin, Dujon and Nicolas (1995) Induction of homologous recombination in mammalian chromosomes by using the I-SceI system of Saccharomyces cerevisiae. Mol Cell Biol, 15, 1968-73.
Christian, Cermak, Doyle, Schmidt, Zhang, Hummel, Bogdanove and Voytas (2010) Targeting DNA Double-Strand Breaks with TAL Effector Nucleases. Genetics 186: 757-761.
Cohen-Tannoudji, Robine, Choulika, Pnto, E1 Marjou, Babinet, Louvard and Jaisser (1998) I-SceI-induced gene replacement at a natural locus in embryonic stem cells. Mol Cell Biol, 18, 1444-8.
Donoho, Jasin and Berg (1998) Analysis of gene targeting and intrachromosomal homologous recombination stimulated by genomic double-strand breaks in mouse embryonic stem cells. Mol Cell Biol, 18, 4070-8.
Dujon, Colleaux, Jacquier, Michel and Monteilhet (1986) Mitochondrial introns as mobile genetic elements: the role of intron-encoded proteins. Basic Life Sci, 40, 5-27.
Gouble, Smith, Bruneau, Perez, Guyot, Cabaniols, Leduc, Fiette, Ave, Micheau, Duchateau and Paques (2006) Efficient in toto targeted recombination in mouse liver by meganuclease-induced double-strand break. J Gene Med, 8, 616-22.
Haber (1995) In vivo biochemistry: physical monitoring of recombination induced by site-specific endonucleases. Bioessays, 17, 609-20.
Hinnen, Hicks and Fink (1978) Transformation of yeast. Proc Natl Acad Sci USA, 75, 1929-33.
Dong-Il Jin, Seung-Hyeon Lee, Jin-Hee Choi, Jae-Seon Lee, Jong-Eun Lee, Kwang-Wook Park and Jeong-Sun Seo (2006) Targeting efficiency of alpha-1,3-galactosyl transferasegene in pig fetal fibroblast cells EMM 35(6), 2003; 572
Kim, Cha, Chandrasegaran (1996). Hybrid restriction enzymes: zinc finger fusions to Fok I cleavage domain. Proc Natl Acad Sci USA 93 (3): 1156-60.
Khanahmad, Noori Daloii, Shokrgozar, Azadmanesh, Niavarani, Karimi, Rabbani, Khalili, Bagheri, Maryami, Zeinali (2006) A novel single step double positive double negative selection strategy for β-globin gene replacement Biochemical and Biophysical Research Communications no. 1, 14-20.
Perez C, Guyot V, Cabaniols J, Gouble A, Micheaux B, Smith J, Leduc S, Paques F, Duchateau P, (2005) BioTechniques vol. 39, n^{o}1, pp. 109-115
Posfai, Kolisnychenko, Bereczki and Blattner (1999) Markerless gene replacement in Escherichia coli stimulated by a double-strand break in the chromosome. Nucleic Acids Res, 27, 4409-15.
Puchta, Dujon and Hohn (1996) Two different but related mechanisms are used in plants for the repair of genomic double-strand breaks by homologous recombination. Proc Natl Acad Sci USA, 93, 5055-60.
Rothstein (1983) One-step gene disruption in yeast. Methods Enzymol, 101, 202-11.
Rouet, Smih and Jasin (1994) Introduction of double-strand breaks into the genome of mouse cells by expression of a rare-cutting endonuclease. Mol Cell Biol, 14, 8096-106.
Sargent, R. G., Brenneman, M. A., and Wilson, J. H. (1997) Repair of site-specific double-strand breaks in a mammalian chromosome by homologous and illegitimate recombination. Mol Cell Biol, 17, 267-77.
Siebert and Puchta (2002) Efficient Repair of Genomic Double-Strand Breaks by Homologous Recombination between Directly Repeated Sequences in the Plant Genome. Plant Cell, 14, 1121-31.
Smithies (2001) Forty years with homologous recombination. Nat Med, 7, 1083-6.
Thomas and Capecchi (1987) Site-directed mutagenesis by gene targeting in mouse embryo-derived stem cells. Cell, 51, 503-12.

1. A set of genetic constructs, comprising:
a) a construct (i) encoded by a nucleic acid molecule, the construct (i) comprising
(N)ₙ-HOMO1-P-M-HOMO2-(N)ₘ (i),
wherein:
n and m are an integer and represent 0 or 1, with the proviso that when n=1, m=0 and when n=0, m=1;
component N is optionally disposed either before HOMO1 or after HOMO2, and components P and M are optionally disposed in the order P-M or M-P;
N comprises the components (PROM1)-(NEG)-(TERM 1);
P comprises the components (PROM2)-(POS)-(TERM2);
M comprises the components (PROM3)-(MCS)-(TERM3);
PROM 1 is a first transcriptional promoting sequence;
NEG is a negative selection marker;
TERM1 is a first transcriptional termination sequence;
HOMO1 is a portion homologous to a genomic portion preceding a nuclease DNA target sequence;
PROM2 is a second transcriptional promoting sequence;
POS is a positive selection marker;
TERM2 is a second transcriptional termination sequence;
PROM3 is a third transcriptional promoting sequence;
MCS is a multiple cloning site;
TERM3 is a third transcriptional termination sequence; and
HOMO2 is a portion homologous to a genomic portion following said nuclease DNA target sequence;
b) at least one construct selected from the group consisting of a construct (ii), a construct (iii) and a sequence (iv),
wherein:
said construct (ii) and construct (iii) are encoded by nucleic acid molecules and comprise:
PROM4-NUC1 (ii);
NUC2 (iii);
said sequence (iv) is an isolated or recombinant protein comprising:
NUC3 (iv);
PROM4 is a fourth transcriptional promoting sequence;
NUC1 is the open reading frame (ORF) of a meganuclease, TALEN or a ZFN;
MEGA2 is a messenger RNA (mRNA) version of said meganuclease, said TALEN or said ZFN;
MEGA3 is an isolated or recombinant protein of said meganuclease, said TALEN or said ZFN;
said meganuclease, said TALEN or said ZFN from constructs (ii) or (iii) or sequence (iv) recognize and cleave said nuclease DNA target sequence; and
constructs (ii) or (iii) or sequence (iv) are configured to be co-transfected with construct (i) into at least one target cell.

2. The set of constructs of claim 1, wherein said HOMO1 and HOMO2 comprise at least 200 bp and no more than 6000 bp of sequence homologous to the portions of a target cell genome flanking said nuclease DNA target sequence.

3. The set of constructs of claim 1, wherein HOMO1 and HOMO2 comprise at least 1000 bp and no more than 2000 bp of sequence homologous to the portions of a target cell genome flanking said nuclease DNA target sequence.

4. The set of constructs of claim 1, wherein said POS is selected from the group consisting of:
neomycin phosphotransferase resistant gene, hph (SEQ ID NO 3);
hygromycin phosphotransferase resistant gene, hph (SEQ ID NO 4);
puromycin N-acetyl transferase gene, pac (SEQ ID NO 5);
blasticidin S deaminase resistant gene, bsr (SEQ ID NO 6); and
bleomycin resistant gene, sh ble (SEQ ID NO 7).

5. The set of constructs of claim 1, wherein said NEG is selected from the group consisting of:
Thymidine kinase gene of the herpes simplex virus deleted of CpG islands, HSV TK DelCpG (SEQ ID NO 8); and
cytosine deaminase coupled to uracyl phosphoribosyl transferase gene deleted of CpG islands, CD:UPRT DelCpG (SEQ ID NO 9).

6. The set of constructs of claim 1, wherein said elements PROM1, PROM2, PROM3 and PROM4 are selected from the group consisting of:
cytomegalovirus immediate-early promoter, pCMV (SEQ ID NO 10);
simian virus 40 promoter, pSV40 (SEQ ID NO 11);
human elongation factor 1α promoter, phEF 1α (SEQ ID NO 12);
human phosphoglycerate kinase promoter, phPGK (SEQ ID NO 13);
murine phosphoglycerate kinase promoter, pmPGK (SEQ ID NO 14);
human polyubiquitin promoter, phUbc (SEQ ID NO 15);
thymidine kinase promoter from human herpes simplex virus, pHSV-TK (SEQ ID NO 16);
human growth arrest specific 5 promoter, phGAS5 (SEQ ID NO 17);
tetracycline-responsive element, pTRE (SEQ ID NO18);
internal ribosomal entry site (IRES) sequence from encephalopathy myocarditis virus, IRES EMCV (SEQ ID NO 19); and
IRES sequence from foot and mouth disease virus, IRES FMDV (SEQ ID NO 20), SV40.

7. The set of constructs of claim 1, wherein said elements TERM1, TERM2, TERM3 and TERM4 are selected from the group consisting of:
polyadenylation signal, SV40 pA (SEQ ID NO 21); and
bovine growth hormone polyadenylation signal, BGH pA (SEQ ID NO 22).

8. The set of constructs of claim 1, wherein said MCS comprises an in frame peptide tag at its 5′ or 3′ end, wherein said peptide tag is selected from the group consisting of FLAG (SEQ ID NO 23), FLASH/REASH (SEQ ID NO 24), IQ (SEQ ID NO 25), histidine (SEQ ID NO 26), STREP (SEQ ID NO 27), streptavidin binding protein, SBP (SEQ ID NO 28), calmodulin binding protein, CBP (SEQ ID NO 29), haemagglutinin, HA (SEQ ID NO 30), c-myc (SEQ ID NO 31), V5 tag sequence (SEQ ID NO 32), nuclear localization signal (NLS) from nucleoplasmin (SEQ ID NO 33), NLS from SV40 (SEQ ID NO 34), NLS consensus (SEQ ID NO 35), thrombin cleavage site (SEQ ID NO 36), P2A cleavage site (SEQ ID NO 37), T2A cleavage site (SEQ ID NO 38), and E2A cleavage site (SEQ ID NO 39).

9. The set of constructs of claim 1, wherein said MCS comprises a reporter gene selected from the group consisting of firefly luciferase gene (SEQ ID NO 40), renilla luciferase gene (SEQ ID NO 41), β-galactosidase gene, LacZ (SEQ ID NO 42), human secreted alkaline phosphatase gene, hSEAP (SEQ ID NO 43), murine secreted alkaline phosphatase gene, and mSEAP (SEQ ID NO 44).

10. The set of genetic constructs of claim 1, wherein construct (i) comprises SEQ ID NO: 45 or SEQ ID NO: 46.

11. A kit to introduce a sequence encoding a GOI into at least one cell, the kit comprising:
the set of genetic constructs of claim 1; and
instructions for generating a transformed cell with said set of genetic constructs.

12. The kit of claim 11, further comprising:
at least one target cell is selected from the group consisting of CHO-K1 cells, HEK293 cells, Caco2 cells, 30 U2-OS cells, NIH 3T3 cells, NSO cells, SP2 cells, CHO-S cells, and DG44 cells.

13. A method for transforming by homologous recombination at least one cell, the method comprising:
a) cloning a sequence coding for a gene into position MCS of a construct (i);
b) co-transfecting a target cell with said construct (i) and at least one of a construct (iii), a construct (ii) or a sequence (iv);
c) selecting at least one cell based upon the presence of a POS and the absence of an NEG from said target cell,
wherein:
said construct (i) is encoded by a nucleic acid molecule and comprises:
(N)ₙ-HOMO1-P-M-HOMO2-(N)ₘ (i),
wherein:
n and m are an integer and represent 0 or 1, with the proviso that when n=1, m=0 and when n=0, m=1;
component N is optionally disposed either before HOMO1 or after HOMO2, and components P and M are optionally disposed in the order P-M or M-P;
N comprises the components (PROM1)-(NEG)-(TERM 1);
P comprises the components (PROM2)-(POS)-(TERM2);
M comprises the components (PROM3)-(MCS)-(TERM3);
PROM1 is a first transcriptional promoting sequence;
NEG is a negative selection marker;
TERM1 is a first transcriptional termination sequence;
HOMO1 is a portion homologous to a genomic portion preceding a nuclease DNA target sequence;
PROM2 is a second transcriptional promoting sequence;
POS is a positive selection marker;
TERM2 is a second transcriptional termination sequence;
PROM3 is a third transcriptional promoting sequence;
MCS is a multiple cloning site;
TERM3 is a third transcriptional termination sequence; and
HOMO2 is a portion homologous to a genomic portion following said nuclease DNA target sequence;
said construct (ii) and construct (iii) are encoded by nucleic acid molecules and comprise:
PROM4-NUC1 (ii);
NUC2 (iii);
said sequence (iv) is an isolated or recombinant protein comprising:
NUC3 (iv),
wherein:
PROM4 is a fourth transcriptional promoting sequence;
NUC1 is the open reading frame (ORF) of a meganuclease, TALEN or a ZFN;
MEGA2 is a messenger RNA (mRNA) version of said meganuclease, said TALEN or said ZFN;
MEGA3 is an isolated or recombinant protein of said meganuclease, said TALEN or said ZFN;
said meganuclease, said TALEN or said ZFN from constructs (ii) or (iii) or sequence (iv) recognize and cleave said nuclease DNA target sequence; and
constructs (ii) or (iii) or sequence (iv) are configured to be co-transfected with construct (i) into at least one target cell.

14. The method of claim 13, wherein selection c) is carried out sequentially for the activity of the gene product encoded by POS and NEG.

15. The method of claim 13, wherein selection in step c) is carried out simultaneously for the activity of the gene product encoded by POS and NEG.

16. The set of constructs of claim 2, wherein HOMO1 and HOMO2 comprise at least 1000 bp and no more than 2000 bp of sequence homologous to the portions of a target cell genome flanking said nuclease DNA target sequence.

17. A kit to introduce a sequence encoding a GOI into at least one cell, the kit comprising:
the set of genetic constructs of claim 2; and
instructions for generating a transformed cell with said set of genetic constructs.

18. The kit of claim 17, further comprising:
at least one target cell is selected from the group consisting of CHO-K1 cells, HEK293 cells, Caco2 cells, 30 U2-OS cells, NIH 3T3 cells, NSO cells, SP2 cells, CHO-S cells, and DG44 cells.

19. The method of claim 13, wherein HOMO1 and HOMO2 comprise at least 1000 bp and no more than 2000 bp of sequence homologous to the portions of a target cell genome flanking said nuclease DNA target sequence.
